(19) United States
(12) Patent Application Publication
(10) Pub. No.: US 2004/0120964 A1
(43) Pub. Date: Jun. 24, 2004

(54) Needleless vaccination using chimeric yellow fever vaccine-vectored vaccines against heterologous flaviviruses

(76) Inventors: John A. Mikszta, Durham, NC (US); Jason B. Alarcon, Durham, NC (US); Cheryl Dean, Raleigh, NC (US); Andrea Waterston, Holly Springs, NC (US); Farshad Guirakhoo, Melrose, ME (US); Monath P. Thomas, Harvard, MA (US)

Correspondence Address: JONES DAY, 222 EAST 41ST ST, NEW YORK, NY, 10017, US

(21) Appl. No.: 10/679,036

(22) Filed: Oct. 2, 2003

Related U.S. Application Data
(63) Continuation-in-part of application No. 10/282,231, filed on Oct. 29, 2002.
(60) Provisional application No. 60/330,713, filed on Oct. 29, 2001.
(60) Provisional application No. 60/333,162, filed on Nov. 27, 2001.

(51) Int. Cl. A61K 39/00; A61K 39/38

(52) U.S. Cl. 424/184.1; 604/46; 206/367

(57) ABSTRACT

The present invention relates to a method of vaccinating a subject comprising delivering a chimeric yellow fever 17D strain vector expressing an envelope protein gene product of a heterologous flavivirus to the epidermal compartment or the intradermal compartment of the subject's skin. The invention encompasses vaccine compositions comprising the chimeric yellow fever viruses expressing an envelope protein gene product of a heterologous flavivirus. The compositions of the invention result in an enhanced therapeutic efficacy, e.g., enhanced protective immune response as they enhance the presentation and availability of the chimeric vaccine to the targeted compartment of the subject's skin.

This application claims the benefit of priority of U.S. application Ser. No. 10/282,231, filed on Oct. 29, 2002, U.S. Provisional Application Nos. 60/330,713 and 60/333,162, filed Oct. 29, 2001 and Nov. 27, 2001, respectively, which are incorporated herein by reference in their entireties.

### 1. FIELD OF THE INVENTION

The present invention relates to a method of vaccinating a subject comprising delivering a chimeric yellow fever 17D strain vector expressing an envelope protein gene product of a heterologous flavivirus to the epidermal compartment or the intradermal compartment of the subject's skin. The invention encompasses vaccine compositions comprising the chimeric yellow fever viruses expressing an envelope protein gene product of a heterologous flavivirus. The vaccine compositions of the invention result in an enhanced therapeutic efficacy, e.g., enhanced protective immune response as they enhance the presentation and availability of the chimeric vaccine to the targeted compartment of the subject's skin.

### 2. BACKGROUND OF THE INVENTION

2.1 Flaviviruses

The family Flaviviridae includes approximately 60 enveloped, positive strand RNA viruses, most of which are transmitted by an insect vector. Most flaviviruses are arthropod-borne, being transmitted to vertebrates by chronically infected mosquito or tick vectors. Arthropod-borne flaviviruses cause significant human and animal diseases and are distributed world wide. Many members of this family cause significant public health problems in different regions of the world (Monath, T. P. 1986 In: The Togaviridae and Flaviviridae, S. Schlesinger et al., eds. pp. 375-440. Plenum Press, New York). Entities of major global concern include Dengue fever with its associated dengue hemorrhagic fever (DHF) and shock syndrome (DSS), Japanese Encephilitis (JE). Tick-borne encephilitis (TBE), Kyasanur Forest disease, West Nile encephilitis, St. Louis encephilitis, and Murray Valley Encephilitis are other important agents of regional endemic or epidemic disease.

The genome of all flaviviruses sequenced thus far has the same gene order: 5′-C-preM-E-NS1-NS2A-NS2B-NS3-NS4A-NS4B-NS5-3′ in which the first three genes code for the structural proteins, the capsid (C), the premembrane protein (pre M) and the envelope protein (E). Fully processed mature virions of flaviviruses contain three structural proteins, envelope (E), capsid (C), and membrane (M) and seven non-structural proteins (NS1, NS2A, NS2B, NS3, NS4A, NS4B, and NS5). Immature flavivirions found in infected cells contain prM protein, which is the precursor of the M protein. After binding of virions to host cell receptors, the E protein undergoes an irreversible conformational change upon exposure to the acidic pH of the endosomes, causing fusion between the envelope bilayers of the virions and endocytic veiscles, thus releasing the viral genome into the host cytosol. The cleavage of the prM protein to M protein occurs shortly before release of the virions (Stadler et al., 1997, J. Virol. 71: 8475-81), which is needed to activate hemagglutinating activity, fusogenic activity, and infectivity of virions. The M protein is cleaved from its precursor after a consensus sequence, R-X-R/K-R, wherein X is variable and incorporated into the virus lipid envelope together with the E protein.

Several members of the flavivirus family pose current or potential threat to global public health. Dengue is a mosquito-borne viral disease which occurs in tropical and sub-tropical regions throughout the world. The dengue virus subgroup causes more human disease than any other member of the flavivirus family. Dengue is characterized by fever, rash, severe headache and joint pain. Its mortality rate is low. However, over the past few decades, a more severe form of dengue, characterized by hemorrhage and shock (dengue hemorrhagic fever/dengue shock syndrome; DHF/DSS) has been observed with increasing frequency in children and young adults. DHF/DSS occurs most often during dengue virus infection in individuals previously infected with another dengue virus serotype. This has led to the suggestion that immune enhancement of viral replication plays a role in the pathogenesis of the more severe form of disease (Halstead, S. B., 1988 Science 239, 476-481).

Soon after their isolation in 1944, dengue viruses were passaged repeatedly in mouse brain, resulting in the selection of mouse neurovirulent mutants (Sabin, A. B., 1952 Amer. J Trop. Med. Hyg. 1:30-50). Interestingly, studies performed in volunteers showed that mouse brain-adapted neurovirulent mutants of three strains of type 1 or type 2 dengue virus were attenuated, but still immunogenic for humans (Sabin, A. B., 1952 Amer. J. Trop. Med. Hyg. 1:30-50; Sabin, A. B., 1955 Amer. J Trop. Med. Hyg. 4:198-207; Sabin, A. B., 1955 Amer. J. Trop. Med. Hyg. 4:198-207; Schlesinger, R. W. et al., 1956 J. Immunol. 77:352-364; Wisseman, C. L. et al., 1963 Amer. J. Trop. Med. 12:620-623). However, the mutants were not developed further as candidate vaccine strains because of concern for mouse brain antigens in the vaccine preparations. Since that time, virus mutants that: (i) exhibited the small plaque size phenotype, and/or (ii) were temperature sensitive, and/or (iii) were adapted to cell cultures derived from an unnatural host (i.e., host range mutants), have been selected and evaluated as candidates for inclusion in a live attenuated virus vaccine (Harrison, V. R. et al. (1977) Infec. Immun. 18:151-156; Hoke, C. H. et al. (1990) Am. J. Trop. Med. Hyg. 43:219-226; Bhamarapravati, N. et al. (1987) Bull. WHO. 65:189-195). However, despite 25 years of such efforts, safe, effective dengue vaccines are still not available for general use. Inactivated whole dengue virus vaccines have been shown to be insufficiently immunogenic. Live virus vaccines attenuated by serial passage in cell culture have suffered from genetic instability under attenuation or poor immunogenicity. Attenuated dengue viruses generated thus far are genetically unstable and have the potential to revert back to a pathogenic form over time. Yet they are desirable since they are generally known to provide long lasting immunity.

Dengue viruses continue to cause major epidemics throughout the tropical and subtropical regions of the world. Despite many years of research effort, an effective vaccine is not available. The predominant disease associated with dengue viral infection is a debilitating illness known as dengue fever. Less frequently, dengue virus causes a hemorrhagic shock syndrome in young children, which has a very high mortality rate. Thus, control of dengue fever and dengue hemorrhagic shock is a major global concern. Consequently, the WHO has designated the dengue viruses as one of five high priority targets for accelerated vaccine development. The industry is lacking a vaccine formed from a genetically engineered dengue protein.

Other members of the Flavivirus family are also pathogenic. Examples include tick-borne encephalitis virus and Japanese Encephalitis Virus. Tick-borne encephalitis virus (TBEV) is transmitted exclusively by ticks and can be divided into two serologically distinguishable subtypes: the Eastern subtype (prototype strain Sofjin), prevalent in Siberian and Far Eastern regions of Russia, and the Western subtype (prototype strain Neudorfl), common in eastern and central Europe. TBEV causes a serious encephalitic illness with a mortality rate ranging from 1 to 30%. For a review of TBEV see Calisher, et al. (J. Gen. Virol 70: 37-43). Currently, an experimental TBE vaccine produced by formalin inactivation of TBEV is available, but this vaccine has several limitations. For example, the vaccine is not sufficiently immunogenic, therefore repeated vaccinations are required to generate a protective immune response. Even when antibody responses to the vaccine are present, the vaccine fails to provide protective responses to the virus in 20% of the population. Therefore, there remains a need for an improved TBEV vaccine.

Like attenuated dengue virus vaccines, attenuated tick-borne encephalitis virus (TBEV) virus has tended to be genetically unstable and poorly immunogenic. Therefore, other attenuated flavivirus vaccines would also be a considerable advance in the art. There have been efforts to modify full-length recombinant cDNA constructs of dengue virus or another flavivirus as a framework for gene manipulation and chimeric virus development for the production of vaccines to other Flaviviruses.

2.2 Vaccine Technologies

The advantages of delivering drugs and vaccines by routes other than injection with syringe and needle have been recognized for many years. The syringe and needle has been implicated in transmission of blood-borne diseases, particularly HIV, hepatitis C and hepatitis C, both through needle-stick injuries of health care workers and through inadvertent or intentional reuse of needles. In addition, needle injection is painful and objectionable to many patients. For these reasons, considerable effort has been expended in finding alternatives to the use of needles for injection. In the case of vaccines, alternatives to needle injection that have been investigated include orally or intranasally administered vaccines and vaccines that are injected through the skin using a mechanical device that generates air pressure sufficiently strong to force a fluid through the epidermis and/or dermis. Unfortunately, there are few examples of vaccines that can be delivered effectively by the oral or intranasal route. As for needle less injection by pressure-generating devices, these have the disadvantage of being relatively complex and expensive, particularly for single-use applications. There are many companies with patented devices that use devices generating high pressure to force liquid vaccines or drugs through the skin, including Medi-ject (Antares Pharma), Syrijet (Keystone Industries), J-Tip (National Medical Products), and a device developed by Powderject for use with both naked DNA (gene gun) and powdered vaccines. The majority of these devices deliver the product into he subcutaneous tissues, the traditional compartment for injection of drugs and vaccines by needle injection. Another approach described by the Iomal Corporation (U.S. Pat. No. 5,980,898) and Alza (U.S. Pat. No. 6,230,051) focuses on transdermal patches for drug or vaccine delivery. The Powderject, Alza and Iomal technologies recognize the importance of inoculation of vaccines into the superficial layers of the skin itself, particularly the epidermis which contains dendritic and Langerhans cells.

The success of any vaccine relies on the presentation of specific antigens to the immune system in order to activate strong and durable immunity. Dendritic cells are the most potent antigen-presenting cells of the immune system. Thus, the efficiency of vaccination is significantly enhanced if antigens are targeted to dendritic cells.

Because of their central role in immunization, dendritic cells have been the focus of attention for the development of vaccines against cancer, which are designed to break tolerance (Chada et al., 2003, Curr. Opin. Drug Discovery Dev. 6(2): 169-73) or as vaccines against autoimmune diseases designed to induce tolerance. Transimmunization is the term used to refer to the transfer of tumor antigens to newly formed dendritic cells capable of initiating immunization against the tumor cells. For these applications autologous dendritic cells derived from blood may be transduced with genes encoding antigens. Alternatively, dendritic cells may be pulsed with peptide antigens and reinfused.

The use of dendritic cells in vaccination strategies against infectious diseases has received much less attention than the application of transimmunization for cancer.

It has only recently been appreciated that the skin is actually the largest immunologically active organ in the body. The skin is the principal barrier between the contaminated environment and sterile tissues of the host. In addition to its physical barrier function, the skin plays a critical role in immunological defense. Langerhans cells are involved in activation of the immune system. Langerhans cells are bone marrow-derived epidermal dendritic cells. When exposed to antigen, these cells migrate from the epidermis through the lymphatics to the draining lymph nodes where they are responsible for the activation of T cells in the primary immune response.

The role of Langerhans cells in the initiation immunity was suggested by Becker (Becker et al., 1994, Virus Genes, 9(1): 33-45). Becker proposed to use synthetic flavivirus peptides with an amino acid motif to fit with the HLA class I peptide binding group of HLA haplotypes prevalent in a given population in an endemic area. These synthetic viral peptides were to be introduced into the human skin using a lotion containing the peptides (“Peplotion”) together with substances capable of enhancing the penetration of these peptides into the skin to reach Langerhans cells. This hypothesis was not reduced to practice and did not recognize that flaviviruses already have the potential to infect Langerhans cells, making delivery of nonreplicating peptides unnecessary.

Flaviviruses, like other arthropod-borne viruses gain access to the body by the bit of blood-feeding mosquitoes or ticks. The site of deposition of mosquito or tick saliva containing virus is the epidermis and dermis. However, the interactions between virus deposited in this fashion and host cells are not well understood. It is logical to assume that viruses inoculated in this fashion encounter Langerhans cells. There are a few reports on the role of Langerhans cells in flavivirus infections and summarized below.

Taweechaisupapong and colleagues studied skin Langerhans cell morphologic changes after infection with dengue virus in nonhuman primates (Taweechaisupapong et al, 1996, Southeast Asian J. Trop. Med. Pub. Health, 27(4): 664-72). The virus was inoculated by the intradermal route. A time-lapse study of skin biopsies at the injection sites, revealed a highly active migration of epidermal Langerhans cells. The same authors also showed that after intradermal injection of mice injection into the footpads of mice, Langerhans cells increased in numbers at the site of injection and that the resulting neutralizing antibody levels were higher than those induced by intramuscular injection (Taweechaisupapong et al., 1996, J. Med. Microbiol. 45(2): 138-45).

Byrne and colleagues studied the interactions of Langerhans cells and another flavivirus, West Nile. They showed that the virus infects Langerhans cells, causes Langerhans cell migration, accumulation in the draining lymph nodes, and altered lymph node function dependent on interleukin-1 beta (Byrne et al., 2001, J. Invest. Dermatol. 117(3): 702-9).

Wu and colleagues showed that Langerhans cells were the principal target cell for replication of dengue virus (6). Initial experiments demonstrated that blood-derived dendritic cells were 10-fold more permissive for DV infection than were monocytes or macrophages. Epidermal Langerhans were confirmed to be permissive for dengue infection. This observation was confirmed by Marovich et al., 2001, J. Invest. Dermatol. Symp. Proc. 6(3): 219-24).

Despite the numerous efforts in developing various modes of delivery for vaccines, there is still an unmet need in delivering flaviviruses, particularly chimeric flaviviruses, while achieving optimal therapeutic efficacy or a protective immune response. The present invention addresses this need by developing methods for delivering chimeric flaviviruses to the epidermal compartment of a subject's skin by needle-less devices and microneedle devices, thereby enhancing the therapeutic efficacy and protective immune responses of the chimeric viruses. The enhanced efficacy of the formulations of the invention is based, in part, on their ability to specifically target the immune cells of the skin, e.g., dendritic cells and Langerhans cells.

### 3. SUMMARY OF THE INVENTION

The present invention is based, in part, on the inventors' surprising discovery that delivering a chimeric flavivirus vaccine formulation to the epidermal compartment of a subject's skin enhances the therapeutic efficacy and protective immune response of the chimeric flavivirus vaccine by specifically targeting the vaccine to the immune cells of the skin, including the dendritic cells and Langerhans cells. The enhanced efficacy of the chimeric vaccine formulations of the invention are based, in part on the recognition and appreciation by the inventors that the dendritic cells and Langerhans cells provide an ideal immunological space for direct access of the chimeric vaccine to the immune cells residing therein. The dendritic cells and Langerhans cells not only allow more efficient replication of the chimeric virus but also allow for a more effective immune response as they are responsible for the activation of T cells in the primary immune response.

The present invention is based, in part, on the discovery that a live attenuated flavivirus vaccine comprising a chimeric yellow fever 17D backbone expressing the envelope genes of a heterologous dengue virus is more infectious in dendritic cells compared to the parental wild type dengue strains. This chimeric vaccine also known an ChimeriVax™, is a live attenuated genetically engineered virus, wherein the genes encoding two structural proteins, the premembrane protein (prM) and envelope proteins (E) of the yellow fever virus (YF) 17D vaccine strain are replaced with the corresponding genes of the target Dengue virus (see, e.g., Guirakhoo et al., 2001, J. Virol. 75(16): 7290-7304). This observation held true for chimeric viruses constructed from all 4 dengue serotypes compared to the parental wild-type dengue strains. The inventors, thus, for the first time, demonstrate, using in vitro and in vivo studies, that dendritic cells are the initial targets of infection by ChimeriVax™ viruses and that presentation of these viruses to dendritic cells in the skin is the most efficient means of immunization.

Although not intending to be bound by a particular mechanism of action, the higher infectivity of the chimeric flaviviruses may be due to better presentation of the ChimeriVax™ to dendritic cells, which may result in induction of a different cytokine milieu that modulates the infection of dendritic cells and a more efficient replication of the chimeric virus in the dendritic cells. Furthermore, the more efficient replication of the chimeric vaccine in the dendritic cells may induce a more robust immune response in the host once it is presented preferentially to dendritic cells. Alternatively, the virulent wild-type virus might shut down macromolecular synthesis of the cells, induce apoptosis, or have another deleterious effect on viral replication.

The chimeric flavivirus vaccine formulations of the invention are live, infectious, attenuated viruses, composed of: (1) a first flavivirus, preferably a yellow fever virus, e.g., strain 17D, which is preferably a live attenuated vaccine virus, in which the nucleotide sequence encoding an envelope protein, e.g., the prM and E protein, is modified, for example by deletion, truncation or mutation so that the functional envelope protein of the first flavivirus is not expressed; (2) a nucleotide sequence encoding the viral envelope protein of a second flavivirus, which is different from the first flavivirus, so that the envelope protein of the second flavivirus is expressed. The invention encompasses chimeric flavivirus vaccines known in the art, such as those disclosed in International Publication No. WO 01/39802, which is incorporated herein by reference in its entirety. The chimeric viruses for use in the methods of the invention thus contain genes and gene products that are responsible for intracellular replication of the first flavivirus, and gene and gene products of the viral envelope of the second flavivirus. Since the viral envelope contains antigenic determinants responsible for inducing neutralizing antibodies, the result of infections with the chimeric virus of the invention is that neutralizing antibodies are generated against the second flavivirus.

Preferred flaviviruses for use as the second flavivirus in the chimeric viruses of the invention, include but are not limited to Japanese Encephalitis (JE, e.g., JE SA14-14-2), Dengue (DEN, e.g., any of the Dengue serotypes 1-4); Murray Valley encephalitis, St Louis Encephalitis, West Nile, Tick borne encephalitis, Hepatitis C viruses, Kunjin virus, Powassan virus, Kyasanur Forest Disease virus, and Omsk Hemorrhagic Fever Virus.

In some embodiments, the invention encompasses methods of delivering the chimeric flavivirus vaccine formulations of the invention by specifically targeting the epidermal compartment of the subject's skin. Delivering the chimeric flavivirus vaccine formulations of the invention in accordance with the methods of the invention preferably induces an immune response, more preferably a protective immune response in the subject. The chimeric flavivirus vaccine formulations of the invention have enhanced efficacy, e.g., enhanced protective immune response, as the antigenic or immunogenic agent is delivered to the epidermal compartment with an enhanced availability and/or presentation to the immune cells that reside therein, e.g., antigen presenting cells. The enhanced efficacy of the chimeric flavivirus vaccine formulations results in a therapeutically effective response, e.g., protective immune response, after a single dose, with lower doses of the antigenic or immunogenic agent than conventionally used, and without the need for booster immunizations.

Although not intending to be bound by a particular mechanism of action administering a live attenuated flavivirus vaccine in accordance with the methods of the invention minimizes the dose required for immunization by targeting the epidermal cells (e.g., dendritic or Langerhans cells) which are responsible for replication of the vaccine virus. Additionally administering a live attenuated flavivirus vaccine in accordance with the methods of the invention maximizes the immune response against the relevant antigens by targeting the epidermal cells (e.g., dendritic or Langerhans cells) responsible for antigen presentation to and activation of effector T cells.

The invention encompasses any method known in the art for delivering a vaccine to the epidermal compartment of a subject's skin, e.g., using a microabrader device, such as those disclosed in U.S. Pat. No. 6,595,947; and U.S. Publication No. 2003/0093040 (U.S. application Ser. No. 10/282,231, filed Oct. 29, 2002); U.S. Provisional Application Nos. 60/330,713 and 60/333,162 filed on Oct. 29, 2001 and Nov. 27, 2001, respectively, all of which are incorporated herein by reference in their entirety. The devices used for epidermal delivery of the chimeric vaccine formulations of the invention may be needleless or they may comprise at least one microneedle. In some embodiments, the method of vaccination includes delivery of the chimeric attenuated virus to the epidermal compartment of a subject's skin, preferably to the dendritic cells, using a device that disrupts the outer barrier layer of the skin, i.e., the stratum corneum. The invention encompasses methods for delivering the attenuated chimeric flavivirus vaccine to the skin of a subject, using devices that abrade the skin, particularly the stratum corneum. Devices and methods for delivery to the skin by abrasion are known in the art and encompassed within the invention, see, e.g., US Publication No. 2003/0093040, which is incorporated herein by reference in its entirety.

In some embodiments, devices used for epidermal delivery of the chimeric vaccines of the invention penetrate, but do not pierce, the stratum corneum. The chimeric vaccines to be administered using the methods of this invention may be applied to the skin prior to abrading, simultaneous with abrading, or post-abrading. In a preferred embodiment of the invention, chimeric flavivirus vaccines of the invention are applied to the skin simultaneously with abrasion rather than being applied to previously abraded skin. Although not intending to be bound by a particular mechanism of action, the efficacy of chimeric flaviviral vaccines are improved when they are abraded into the skin rather than being passively applied to skin which has been previously abraded.

In some embodiments, the invention encompasses delivering the chimeric flavivirus vaccines of the invention to the intradermal compartment of a subject's skin preferably by directly and selectively targeting the intradermal space. Although not intending to be bound by a particular mechanism of action, delivering the vaccine formulations of the invention to the intradermal space improves the availability of the chimeric vaccine to the antigen presenting cells residing therein and enhances the antigen specific immune response. The intradermal vaccine formulations of the invention are administered using any of the intradermal devices and methods disclosed in U.S. patent application Ser. No. 09/417,671, filed on Oct. 14, 1999; Ser. No. 09/606,909, filed on Jun. 29, 2000; Ser. No. 09/893,746, filed on Jun. 29, 2001; Ser. No. 10/028,989, filed on Dec. 28, 2001; Ser. No. 10/028,988, filed on Dec. 28, 2001; or International Publication No.'s EP 10922 444, published Apr. 18, 2001; WO 01/02178, published Jan. 10, 2002; and WO 02/02179, published Jan. 10, 2002; and U.S. application Ser. No. 10/185,717 filed on Jul. 1, 2002, all of which are incorporated herein by reference in their entirety.

The vaccine formulations of the invention may be used in a method for treatment and/or prevention of a flavivirus infection. The subject to which the vaccine or an immunogenic composition of the invention is administered is preferably a mammal, most preferably a human, but can also be a non-human animal, including but not limited to, primates, cows, horses, sheep, pigs, fowl (e.g., chickens, turkeys), goats, cats, dogs, hamsters, mice and rodents.

In some embodiments, the chimeric flavivirus vaccine formulations of the invention further comprise one or more additives, including but not limited to, adjuvants, excipients, stabilizers, and penetration enhancers.

The present invention provides methods of vaccinating a subject with a live attenuated chimeric flavivirus vaccine by administering the vaccine to the epidermal compartment or the in tradermal of the subject's skin. Subjects which may be vaccinated using the methods of the invention include but are not limited to, humans, livestock, and companion animals.

In some embodiments, the invention encompasses a method of simultaneously immunizing a host against multiple live attenuated flavivirus vaccines by targeting different sites of the skin containing epidermal cells with each of the vaccine components, thereby avoiding interference effects that occur when multiple viruses infect the same cell subset. In this application of the invention, chimeric yellow fever 17D-dengue 1, -dengue-2, dengue-3 and dengue-4, either singly or in specified combinations, may be conveniently administered to different areas of the skin (as opposed to being mixed together and injected by conventional syringe and needle). It will be appreciated by one skill in the a14 that this application is equally applicable to intradermal delivery of the chimeric vaccines of the invention.

The methods of the invention are particularly advantageous for developing rapid and high levels of immunity against the antigenic or immunogenic agent of the chimeric flaviviruses, against which an immune response is desired. The chimeric flavivirus vaccine formulations of the invention can achieve a systemic immunity at a protective level with a low dose of the antigenic or immunogenic agent. In some embodiments, the vaccine formulations of the invention result in a protective immune response with a dose of the antigenic or immunogenic agent which is 60%, preferably 50%, more preferably 40% of the dose conventionally used for the antigenic or immunogenic agent in obtaining an effective immune response. In preferred embodiments, the vaccine formulations of the invention comprise a dose of the antigenic or immunogenic agent which is lower than the conventional dose used in the art, utilizing the conventional modes of vaccine delivery, e.g., intramuscular and intravenous. Preferably, the vaccine formulations of the invention result in a therapeutically or prophylactically effective immune response after a single dose.

The vaccine formulations of the instant invention have an enhanced therapeutic efficacy, safety, and toxicity profile relative to currently available formulations. The benefits and advantages imparted by the vaccine formulations of the invention is, in part, due to the particular formulation and their utility in targeting a specific compartment, e.g., epidermal or intradermal, of skin. Preferably, the vaccine formulations of the invention provide a greater and more durable protection, especially for high risk populations that do not respond well to immunization. Preferably, the methods of the invention have minimal or no localized skin infection or adverse reaction at the inoculation site. The immunization induced by targeting of the chimeric vaccine formulations of the invention is stronger than the immunization induced by conventional syringe and needle injection. This is presumably due to the unique targeting of the chimeric vaccine to dendritic cells by means of epidermal delivery or to the antigen presenting cells by means of intradermal delivery.

The invention also provides a pharmaceutical pack or kit comprising an chimeric flavivirus vaccine formulation of the invention and a device for delivery of the chimeric vaccine in accordance with the methods of the invention. In a specific embodiment the invention provides a kit comprising, one or more containers filled with one or more of the components of the chimeric vaccine formulation of the invention. Associated with such container(s) can be a notice in the form prescribed by a governmental agency regulating the manufacture, use or sale of pharmaceuticals or biological products, which notice reflects approval by the agency of manufacture, use or sale for human administration. In a specific embodiment, the invention comprises kits comprising a device for epidermal delivery and a chimeric flavivirus vaccine formulation of the invention as described herein. In another specific embodiment, the invention comprises kits comprising a device for intradermal delivery and a chimeric flavivirus vaccine formulation of the invention as described herein.

### 4. BRIEF DESCRIPTION OF THE FIGURES

FIG. 1 GROWTH OF YF-17D IN PRESENCE OR ABSENCE OF HUMAN DC. Growth of virus from DCs infected with ChimeriVax-DEN(1-4) was compared to YF-17D.

FIG. 2 GROWTH OF CHIMERIVAX™-DEN(1-4) AND PARENTAL DENGUE VIRUS. Growth of ChimeriVax™-DEN(1-4) and their parent dengue WT viruses in presence or absence of DCs. Upper Left Panel (A): Growth of YF/DEN1 and DEN1 PUO359 in human DC (MOI 0.2.). Upper right (B): Growth of YF/DEN 2 and DEN 2 PUO218 in human DC (MOI 0.07). Lower Left (C): Growth of YF/DEN 3 and DEN3 PaH881 in human DC (MOI 0.07). Lower Right (D): Growth of YF/DEN4 and DEN4 1228 in human DC (MOI 0.07).

FIG. 3 STATISTICAL ANALYSIS OF MAGNITUDE (A) AND DURATION (B) OF VIREMIA. Panel A provides the Oneway analysis of magnitude of viremia; and panel B provides the oneway analysis of duration of viremia.

### 5. DETAILED DESCRIPTION OF THE INVENTION

The invention encompasses live, attenuated chimeric flavivirus vaccines for targeted delivery of one or more flaviviral antigens to a subject's skin. The vaccine formulations of the invention are preferably selectively and specifically targeted to a particular compartment of a subject's skin. In some embodiments, the chimeric flavivirus vaccine formulations of the invention are delivered to the epidermal compartment of a subject's skin. In other embodiments, the chimeric flavivirus vaccine formulations of the invention are delivered to the intradermal compartment of a subject's skin. Delivering the chimeric flavivirus vaccine formulations of the invention to a particular compartment of a subject's skin in accordance with the methods of the instant invention enhances the therapeutic efficacy of the vaccine formulation.

Although not intending to be bound by a particular mechanism of action, the enhanced efficacy of the chimeric flavivirus vaccine formulations of the invention when delivered to the epidermal or the intradermal compartment of a subject's skin are based, in part on the recognition and appreciation by the inventors that the dendritic cells and Langerhans cells provide an ideal immunological space for direct access of the chimeric vaccine to the immune cells residing therein. The dendritic cells and Langerhans cells not only allow more efficient replication of the chimeric virus but also allow for a more effective immune response as they are responsible for the activation of T cells in the primary immune response. The chimeric flavivirus vaccine formulations of the invention may enhance cell-mediated and/or humoral mediated immune response. Cell-mediated immune responses that may be modulated by the chimeric flavivirus vaccine formulations of the invention include for example, Th1 or Th2 CD4+ T-helper cell-mediated or CD8+ cytotoxic T-lymphocytes mediates responses.

The chimeric flavivirus vaccine formulations of the invention are live, infectious, attenuated viruses, composed of: (1) a first flavivirus, preferably a yellow fever virus, e.g., strain 17D, which is preferably a live attenuated vaccine virus, in which the nucleotide sequence encoding an envelope protein, e.g., the prM and E protein, is modified, for example by deletion, truncation or mutation so that the functional envelope protein of the first flavivirus is not expressed; (2) a nucleotide sequence encoding the viral envelope protein of a second flavivirus, which is different from the first flavivirus, so that the envelope protein of the second flavivirus is expressed. The invention encompasses chimeric flavivirus vaccines known in the art, such as those disclosed in International Publication No. WO 01/39802, which is incorporated herein by reference in its entirety. The chimeric viruses for use in the methods of the invention thus contain genes and gene products that are responsible for intracellular replication of the first flavivirus, and gene and gene products of the viral envelope of the second flavivirus. Since the viral envelope contains antigenic determinants responsible for inducing neutralizing antibodies, the result of infections with the chimeric virus of the invention is that neutralizing antibodies are generated against the second flavivirus.

Preferred flaviviruses for use as the second flavivirus in the chimeric viruses of the invention, include but are not limited to Japanese Encephalitis (JE, e.g., JE SA14-14-2), Dengue (DEN, e.g., any of the Dengue serotypes 1-4); Murray Valley encephalitis, St Louis Encephalitis, West Nile, Tick borne encephalitis, Hepatitis C viruses, Kunjin virus, Powassan virus, Kyasanur Forest Disease virus, and Omsk Hemorrhagic Fever Virus.

In some embodiments, the chimeric flavivirus vaccine formulations of the invention further comprise one or more additives including, but not limited to, an adjuvant, an excipient, a stabilizer, and a penetration enhancer. In other embodiments, the chimeric flavivirus vaccine formulations of the present invention may further comprise one or more other pharmaceutically acceptable carriers, including any suitable diluent or excipient. Preferably, the pharmaceutically acceptable carrier does not itself induce a physiological response, e.g., an immune response. Most preferably, the pharmaceutically acceptable carrier does not result in any adverse or undesired side effects and/or does not result in undue toxicity. Pharmaceutically acceptable carriers for use in the chimeric flavivirus vaccine formulations of the invention include, but are not limited to, saline, buffered saline, dextrose, water, glycerol, sterile isotonic aqueous buffer, and combinations thereof. Additional examples of pharmaceutically acceptable carriers, diluents, and excipients are provided in Remington's Pharmaceutical Sciences (Mack Pub. Co., N.J., current edition; all of which is incorporated herein by reference in its entirety). In particular embodiments, the chimeric flavivirus vaccine formulation of the invention, may also contain wetting agents, emulsifying agents, or pH buffering agents. The chimeric flavivirus vaccine formulations of the invention can be formulated as a liquid, powder, gel, paste, film suspension or other pharmaceutically acceptable forms known in the art. The chimeric flavivirus vaccine formulations of the invention may be in any form suitable for epidermal or intradermal delivery.

Preferably, the chimeric flavivirus vaccine vaccine formulations of the invention are stable formulations, i.e., undergo minimal to no detectable level of degradation and/or aggregation of the antigenic or immunogenic agent, and can be stored for an extended period of time with no loss in biological activity, e.g., antigenicity or immunogenicity of the antigenic agent.

The chimeric flavivirus vaccine formulations of the present invention can be prepared as unit dosage forms. A unit dosage per vial may contain 0.1 mL to 1 mL, preferably 0.1 to 0.5 mL of the formulation. In some embodiments, a unit dosage form of the chimeric flavivirus vaccine formulations of the invention may contain 50 μL to 100 μL, 50 μL to 200 μL, or 50 μL to 500 μL of the formulation. If necessary, these preparations can be adjusted to a desired concentration by adding a sterile diluent to each vial. The chimeric flavivirus vaccine formulations of the invention are more effective in eliciting the desired immune response, and thus the total volume for delivery may be less than the volume that is conventionally used.

In some embodiments, the invention encompasses methods of delivering the chimeric flavivirus vaccine formulations of the invention by specifically targeting the epidermal compartment of the subject's skin. The invention encompasses any method known in the art for delivering a vaccine to the epidermal compartment of a subject's skin, e.g., using a microabrader device, such as those disclosed in U.S. Pat. No. 6,595,947; and U.S. Publication No. 2003/0093040 (U.S. application Ser. No. 10/282,231, filed Oct. 29, 2002); U.S. Provisional Application Nos. 60/330,713 and 60/333,162 filed on Oct. 29, 2001 and Nov. 27, 2001, all of which are incorporated herein by reference in their entirety. The devices used for epidermal delivery of the chimeric vaccine formulations of the invention may be needleless or they may comprise at least one microneedle. In some embodiments, the method of vaccination includes delivery of the chimeric attenuated virus to the epidermal compartment of a subject's skin, preferably to the dendritic cells, using a device that disrupts the outer barrier layer of the skin, i.e., the stratum corneum. The invention encompasses methods for delivering the attenuated chimeric flavivirus vaccine to the skin of a subject, using devices that abrade the skin, particularly the stratum corneum. Devices and methods for delivery to the skin by abrasion are known in the art and encompassed within the invention, see, e.g., U.S. Publication No. 2003/0093040, which is incorporated herein by reference in its entirety.

In some embodiments, the invention encompasses delivering the chimeric flavivirus vaccines of the invention to the intradermal compartment of a subject's skin preferably by directly and selectively targeting the intradermal space. The chimeric flavivirus vaccine formulations of the invention are administered to the intradermal compartment of a subject's skin such that the intradermal space of the subject's skin is penetrated, without passing through it. Preferably, the chimeric flavivirus vaccine formulations are administered to the intradermal space at a depth of about 1.0 to 3.0 mm, most preferably at a depth of 1.0 to 2.0 mm. The chimeric flavivirus vaccine formulations of the invention for intradermal delivery provide a pain-free and less invasive mode of administration as compared to conventional modes of administrations, e.g., i.m., for vaccine formulations, and therefore are more advantageous, for example, in terms of the subjects' compliance.

The intradermal vaccine formulations of the invention are administered using any of the intradermal devices and methods disclosed in U.S. patent application Ser. No. 09/417,671, filed on Oct. 14, 1999; Ser. No. 09/606,909, filed on Jun. 29, 2000; Ser. No. 09/893,746, filed on Jun. 29, 2001; Ser. No. 10/028,989, filed on Dec. 28, 2001; Ser. No. 10/028,988, filed on Dec. 28, 2001; or International Publication No.'s EP 10922 444, published Apr. 18, 2001; WO 01/02178, published Jan. 10, 2002; and WO 02/02179, published Jan. 10, 2002; and U.S. application Ser. No. 10/717,187 filed Jul. 2, 2002, all of which are incorporated herein by reference in their entirety.

The chimeric flavivirus vaccine formulations of the invention have little or no short term and/or long term toxicity when administered in accordance with the methods of the invention. Most preferably, the chimeric flavivirus vaccine formulations of the invention when intradermally administered have little or no adverse or undesired reaction at the site of the injection, e.g., skin irritation, swelling, rash, necrosis, skin sensitization.

The chimeric flavivirus vaccine formulations of the invention may be used in a method for treatment and/or prevention of a flavivirus infection. The subject to which the vaccine or an immunogenic composition of the invention is administered is preferably a mammal, most preferably a human, but can also be a non-human animal, including but not limited to, primates, cows, horses, sheep, pigs, fowl (e.g., chickens, turkeys), goats, cats, dogs, hamsters, mice and rodents.

The methods of the invention are particularly advantageous for developing rapid and high levels of immunity against the antigenic or immunogenic agent of the chimeric flaviviruses, against which an immune response is desired. The chimeric flavivirus vaccine formulations of the invention can achieve a systemic immunity at a protective level with a low dose of the antigenic or immunogenic agent. In some embodiments, the vaccine formulations of the invention result in a protective immune response with a dose of the antigenic or immunogenic agent which is 60%, preferably 50%, more preferably 40% of the dose conventionally used for the antigenic or immunogenic agent in obtaining an effective immune response. In preferred embodiments, the vaccine formulations of the invention comprise a dose of the antigenic or immunogenic agent which is lower than the conventional dose used in the art, utilizing the conventional modes of vaccine delivery, e.g., intramuscular and intravenous. Preferably, the vaccine formulations of the invention result in a therapeutically or prophylactically effective immune response after a single dose.

The vaccine formulations of the instant invention have an enhanced therapeutic efficacy, safety, and toxicity profile relative to currently available formulations. The benefits and advantages imparted by the vaccine formulations of the invention is, in part, due to the particular formulation and their utility in targeting a specific compartment, e.g., epidermal or intradermal, of skin. Preferably, the vaccine formulations of the invention provide a greater and more durable protection, especially for high risk populations that do not respond well to immunization. Preferably, the methods of the invention have minimal or no localized skin infection or adverse reaction at the inoculation site. The immunization induced by targeting of the chimeric vaccine formulations of the invention is stronger than the immunization induced by conventional syringe and needle injection. This is presumably due to the unique targeting of the chimeric vaccine to dendritic cells by means of epidermal delivery or to the antigen presenting cells by means of intradermal delivery.

The invention also provides a pharmaceutical pack or kit comprising an chimeric flavivirus vaccine formulation of the invention and a device for delivery of the chimeric vaccine in accordance with the methods of the invention. In a specific embodiment the invention provides a kit comprising, one or more containers filled with one or more of the components of the chimeric vaccine formulation of the invention. Associated with such container(s) can be a notice in the form prescribed by a governmental agency regulating the manufacture, use or sale of pharmaceuticals or biological products, which notice reflects approval by the agency of manufacture, use or sale for human administration. In a specific embodiment, the invention comprises kits comprising a device for epidermal delivery and a chimeric flavivirus vaccine formulation of the invention as described herein. In another specific embodiment, the invention comprises kits comprising a device for intradermal delivery and a chimeric flavivirus vaccine formulation of the invention as described herein.

5.1 Recombinant Chimeric Flavivirus Constructs

The present invention encompasses recombinant chimeric viruses encoded by viral vectors derived from genomes of Yellow Fever Virus and one or more other Flaviviruses. The chimeric flavivirus vaccines of the invention are preferably live, infectious, attenuated virus, composed of: (1) a first flavivirus, preferably a yellow fever virus, e.g., strain 17D, which is preferably a live attenuated vaccine virus, in which the nucleotide sequence encoding an envelope protein, e.g., the prM and E protein, is modified, for example by deletion, truncation or mutation so that the functional envelope protein of the first flavivirus is not expressed; (2) a nucleotide sequence encoding the viral envelope protein of a second flavivirus, i.e., Dengue virus, which is different from the first flavivirus, so that the envelope protein of the second flavivirus is expressed. The invention encompasses chimeric flavivirus vaccines known in the art, such as those disclosed in International Publication No. WO 01/39802, which is incorporated herein by reference in its entirety. In a preferred embodiment, the prM and E protein nucleotide encoding sequence of YF is replaced with the prM and E protein nucleotide encoding sequence of a second flavivirus.

A preferred live virus for use as the first yellow fever in the chimeric vaccines of the invention is YF 17D, see, e.g,. Smithburn et al., Yellow Fever Vaccination, World Health Org. p. 238, 1956; Freestone, in Plotkin et al., eds, Vaccines, 2d ed, W. B, Saunders, Pa., 1995). YF has been studied at the genetic level (Rice et al. 1985, Science 229, 726-33). Other YF strains that may be used in the chimeric vaccine formulations of the invention include but are not limited to, YF 17 DD (Genbank Accession No. U17066); YF-17D213 (Genbank Accession No. U17067), YF 17D-204 France (Genbank Accession No. X15067, X15062), and YF 17D-204, 234 US (Rice et al., 1985, Science 229, 726-33; Rice et al., New Biologist, 1: 285-96; Genbank Accession No. C03700; K 02749). Other Yellow fever strains encompassed within the invention are described by Gallery et al., 1998, Vaccine, 16: 1024-28, which is incorporated herein by reference in its entirety.

Preferred flaviviruses for use as the second flavivirus in the chimeric viruses of the invention, include but are not limited to Japanese Encephalitis (JE, e.g., JE SA14-14-2), Dengue (DEN, e.g., any of the Dengue serotypes 1-4); Murray Valley encephalitis (MVE), St Louis Encephalitis (SLE), West Nile (WN), Tick borne encephalitis (TBE), Hepatitis C viruses, Kunjin virus, Powassan virus, Kyasanur Forest Disease virus, and Omsk Hemorrhagic Fever Virus.

In some embodiments, the second flavivirus envelope protein nucleotide encoding sequence in the chimeric flavivirus vaccines is derived from two different second flaviviruses, e.g., two Dengue strains. In some embodiments, the second flavivirus envelope protein nucleotide encoding sequence in the chimeric flavivirus vaccines is attenuated using methods known to one skilled in the art. When the second flavivirus is a neurotropic virus, such as Japanese Encephalitis, Murray Valley encephalitis (MVE), St Louis Encephalitis (SLE), the envelope protein nucleotide encoding sequence is preferably attenuated. In the case of non-neurotropic viruses, e.g., dengue virus, it may be preferable to use envelope protein nucleotide encoding sequences that are not attenuated. Although not intending to be bound by a particular mechanism of action, maintenance of native sequences may lead to enhanced immunogenicity, and thus a more effective vaccine.

In a preferred embodiment, the second flavivirus envelope protein nucleotide encoding sequence in the chimeric flavivirus vaccines is derived from two different second flaviviruses, e.g., two different Dengue strains. In some embodiments, the second flavivirus envelope protein nucleotide encoding sequence in the chimeric flavivirus vaccines is attenuated using methods known to one skilled in the art. In the case of non-neurotropic viruses, e.g. dengue virus, it may be preferable to use envelope protein nucleotide encoding sequences that are not attenuated. Although not intending to be bound by a particular mechanism of action, maintenance of native sequences may lead to enhanced immunogenicity, and thus a more effective vaccine.

The chimeric flavivirus vaccines of the invention are constructed using common recombinant DNA methodologies known to one skilled in the art. Preferably, the chimeric flavivirus vaccines of the invention are constructed in accordance with methods described in International Publication No. WO 01/39802, which is incorporated herein by reference in its entirety. In particular, the chimeric flavivirus vaccines of the invention are constructed using the principles set forth in International Publication No. WO 01/39802, ensuring proper proteolytic processing of the polypeptide. YF viral proteins are produced by translation of a single long open reading frame to generate a polyprotein. A complex series of post-translational proteolytic processing coupled with host and viral proteases generate the mature viral proteins. The structural proteins are arranged in the order C-prM-E, where C is cased, prM is a precursor of the viral envelope bound M protein, and E is the envelope protein. A stretch of about 20 amino acids separates C-prM and is referred to as the prM signal sequence which is bound by the two proteolytic sites resulting in the release of the C and prM proteins. The amino terminus of prM is generated by host signalase cleavage within the lumen of the ER, and the NS2B-NS3 protease complex is responsible for mediating cleavage at the C terminus of the C protein. Maintenance of a coordinated cleavage of NS2B-NS3 protease complex at the C-terminus of the C protein and the signalase at the N terminus of the prM protein is critical for proper processing of the polyprotein. In particular, in the chimeric vaccines of the invention the length of the prM signal sequence separating the two cleavage sites is substantially maintained to ensure proper processing and subsequent viability of the chimeric viruses. In most preferred embodiments, in constructing the chimeric flavivirus of the invention the prM signal of the YF backbone is maintained. Preferably, when a sequence from the second flavivirus is introduced into the YF backbone, it is inserted after prM signal sequence. Preferably, the length and sequence of the YF prM signal is maintained. In some embodiments, the YF prM signal sequence may be modified by conservative amino acid substitutions.

The expression products and/or recombinant or chimeric virions obtained in accordance with the invention may advantageously be utilized in vaccine formulations. The expression products and chimeric virions of the present invention are particularly useful for vaccines to create vaccines for the protection of a subject from infections with flaviviral infections.

5.2 Vaccine Formulations

The invention encompasses vaccine formulations comprising the recombinant chimeric flaviviruses of the invention to confer protection against infection by a flavivirus, including but not limited to Japanese Encephalitis (JE, e.g., JE SA14-14-2), Dengue (DEN, e.g., any of the Dengue serotypes 1-4); Murray Valley encephalitis (MVE), St Louis Encephalitis (SLE), West Nile (WN), Tick borne encephalitis (TBE), Hepatitis C viruses, Kunjin virus, Powassan virus, Kyasanur Forest Disease virus, and Omsk Hemorrhagic Fever Virus. In other embodiments, the vaccine formulations of the invention confer protection against viruses other than flaviviruses. The vaccine formulations of the invention may be used in a method for treatment and/or prevention of a flavivirus infection. The subject to which the vaccine or an immunogenic composition of the invention is administered is preferably a mammal, most preferably a human, but can also be a non-human animal, including but not limited to, primates, cows, horses, sheep, pigs, fowl (e.g., chickens, turkeys), goats, cats, dogs, hamsters, mice and rodents.

The vaccine formulations of the instant invention have an enhanced therapeutic efficacy, safety, and toxicity profile relative to currently available formulations. The benefits and advantages imparted by the vaccine formulations of the invention is, in part, due to the particular formulation and their utility in targeting the epidermal compartment of skin. Preferably, the vaccine formulations of the invention provide a greater and more durable protection, especially for high risk populations that do not respond well to immunization. Preferably, the methods of the invention have minimal or no localized skin infection or adverse reaction at the inoculation site. The immunization induced by epidermal targeting of the chimeric vaccine formulations of the invention is stronger than the immunization induced by conventional syringe and needle injection. This is presumably due to the unique targeting of the chimeric vaccine to dendritic cells by means of epidermal delivery.

In some embodiments, the vaccine formulations of the invention may be used as a primary prophylactic agent for a subject at risk of flavivirus infection. Alternatively, the vaccine formulations of the invention may be used as a secondary agent for treating a flavivirus-infected subject by stimulating an immune response against the flavivirus.

The vaccine formulations of the invention are preferably, live, attenuated, chimeric viruses containing a YF virus backbone and one or more heterologous sequence from a second flavivirus, e.g., Dengue virus.

The vaccine formulations of the invention can be administered alone or in combination with other vaccines. In some embodiments, a vaccine formulation of the invention is administered in combination with other vaccines or immunogenic formulations that provide protection against the virus to which an immune response is desired.

In some embodiments, the chimeric vaccine formulations of the invention may be used to immunize a subject against one virus, e.g., JE, and later to re-immunize the same subject against a second or third virus. Although not intending to be bound by a particular mechanism of action, the chimeric yellow fever based construct of the invention will not elicit a strong immunity to itself. Prior immunity to yellow fever does not preclude the use of chimeric vaccine of the invention as a vector for heterologous gene expression. A particular advantage of the chimeric yellow fever based vaccine formulations of the invention is that they can be used in populations that are immune to yellow fever due to prior natural infection or vaccination. Furthermore, the chimeric vaccine formulations of the invention may be used repeatedly, or they may be used to immunize simultaneously with several different construct, including yellow fever chimeras with inserts from for example JE, WN, or SLE.

In some embodiments, the chimeric vaccine formulations of the invention further comprise one or more additives including, but not limited to, an adjuvant, an excipient, a stabilizer, and a penetration enhancer. In other embodiments, the chimeric vaccine formulations of the present invention may further comprise one or more other pharmaceutically acceptable carriers, including any suitable diluent or excipient. Preferably, the pharmaceutically acceptable carrier does not itself induce a physiological response, e.g., an immune response. Most preferably, the pharmaceutically acceptable carrier does not result in any adverse or undesired side effects and/or does not result in undue toxicity. Pharmaceutically acceptable carriers for use in the vaccine formulations of the invention include, but are not limited to, saline, buffered saline, dextrose, water, glycerol, sterile isotonic aqueous buffer, and combinations thereof. Additional examples of pharmaceutically acceptable carriers, diluents, and excipients are provided in Remington's Pharmaceutical Sciences (Mack Pub. Co., N.J., current edition; all of which is incorporated herein by reference in its entirety).

In particular embodiments, the chimeric vaccine formulation of the invention, may also contain wetting agents, emulsifying agents, or pH buffering agents. The chimeric vaccine formulations of the invention can be a solid, such as a lyophilized powder suitable for reconstitution, a liquid solution, a suspension, a tablet, a pill, a capsule, a sustained release formulation, or a powder.

The chimeric vaccine formulations of the invention may be in any form suitable for epidermal delivery or intradermal delivery. The chimeric flavivirus vaccines of the invention may be formulated as a liquid, powder, gel, paste, film, suspension, or other pharmaceutically acceptable form known in the art. Preferably, the chimeric vaccine formulations of the invention are stable formulations, i.e., undergo minimal to no detectable level of degradation and/or aggregation of the antigenic or immunogenic agent, and can be stored for an extended period of time with no loss in biological activity, e.g., antigenicity or immunogenicity of the antigenic agent. The pH of the chimeric vaccine formulations of the inventions should not be equal to the isoelectric point of the antigenic or immunogenic agent in the formulation and may range from about 5.0 to about 7.5, preferably at a pH of 7.0, more preferably at a pH of 7.2.

The chimeric vaccine formulations of the invention are administered in amounts that can be determined by one of skill in the art without undue experimentation. Determination of an effective amount of the vaccine formulation for administration is well within the capabilities of those skilled in the art. An effective dose can be estimated initially from in vitro assays. For example, a dose can be formulated in animal models to achieve an induction of an immunity response using techniques that are well known in the art. One having ordinary skill in the art could readily optimize administration to all animal species based on results described herein. Dosage amount and interval may be adjusted individually. For example, when used as an immunogenic composition, a suitable dose is an amount of the composition that when administered as described above, is capable of eliciting an antibody response. When used as a vaccine, the vaccine or immunogenic formulations of the invention may be administered in about 1 to 3 doses for a 1-36 week period. Preferably, 1 or 2 doses are administered, at intervals of about 2 weeks to about 4 months, and booster vaccinations may be given periodically thereafter. Alternate protocols may be appropriate for individual animals. A suitable dose is an amount of the vaccine formulation that, when administered as described above, is capable of raising an immunity response in an immunized animal sufficient to protect the animal from an infection for at least 4 to 12 months. In general, the amount of the antigen present in a dose ranges from about 1 pg to about 100 mg per kg of host, typically from about 10 pg to about 1 mg, and preferably from about 100 pg to about 1 μg. Suitable dose range will vary with the route of injection and the size of the patient, but will typically range from about 0.1 mL to about 5 mL.

The chimeric vaccine formulations may be administered and formulated for example in the same manner as the yellow fever 17D vaccine, e.g., as a clarified suspension of infected chicken embryo tissue, or a fluid harvested from cell cultures infected with chimeric yellow fever virus. In some embodiments, the chimeric vaccine formulations of the invention are formulated as a sterile aqueous solution containing between 100 and 100,000 infectious units, (e.g., plaque forming units)

The chimeric vaccine formulations of the invention may be prepared as unit dosage forms. A unit dosage per vial may contain 0.1 mL to 1 mL, preferably 0.1 to 0.5 mL of the formulation. In some embodiments, a unit dosage form of the vaccine formulations of the invention may contain 50 μL to 100 μL, 50 μL to 200 μL, or 50 μL to 500 μL of the formulation. If necessary, these preparations can be adjusted to a desired concentration by adding a sterile diluent to each vial. The vaccine formulations of the invention are more effective in eliciting the desired immune response, and thus the total volume for delivery may be less than the volume that is conventionally used.

The invention also provides vaccine formulations that are packaged in a hermetically sealed container such as an ampoule or a sachette indicating the quantity of the components. In one embodiment, the vaccine formulation is supplied as a liquid, in another embodiment, as a dry sterilized lyophilized powder or water free concentrate in a hermetically sealed container and can be reconstituted, e.g., with water or saline to the appropriate concentration for administration to a subject. In an alternative embodiment, the vaccine formulation is supplied in liquid form in a hermetically sealed container indicating the quantity and concentration of the components. The vaccine formulation of the invention may be prepared by any method that results in a stable, sterile, injectable formulation.

A vaccine or immunogenic formulation of the invention may be administered to a subject in the form of a pharmaceutical or therapeutic composition. Formulations of the invention may be manufactured by means of conventional mixing, dissolving, granulating, dragee-making, levigating, emulsifying, encapsulating, entrapping or lyophilizing processes. Compositions of the invention may be formulated in conventional manner using one or more physiologically acceptable carriers, diluents, excipients or auxiliaries which facilitate processing of the immunogenic antigen of the invention into preparations which can be used pharmaceutically.

When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum hydroxide, aluminum phosphate gel, Freund's Complete Adjuvant, Freund's Incomplete Adjuvant, squalene or squalane oil-in-water adjuvant formulations, biodegradable and biocompatible polyesters, polymerized liposomes, triterpenoid glycosides or saponins (e.g., QuilA and QS-21, also sold under the trademark STIMULON, ISCOPREP), N-acetyl-muramyl-L-threonyl-D-isoglutamine (Threonyl-MDP, sold under the trademark TERMURTIDE), LPS, monophosphoryl Lipid A (3D-MLAsold under the trademark MPL).

5.3 Administration of Vaccine Formulations

The invention encompasses methods of delivering the chimeric flavivirus vaccine formulations of the invention by specifically targeting the epidermal compartment of the subject's skin. Preferably, the methods of the invention employ needleless devices. The invention encompasses any method known in the art for delivering a vaccine to the epidermal compartment of a subject's skin, e.g., using microabrader device, such as those disclosed in U.S. Pat. No. 6,595,947 and U.S. Publication No. 2003/0093040 (U.S. application Ser. No. 10/282,231, filed Oct. 29, 2002); U.S. Provisional Application Nos. 60/330,713 and 60/333,162 filed on Oct. 29, 2001 and Nov. 27, 2001, respectively, all of which are incorporated herein by reference in their entirety

In some embodiments, the invention encompasses delivering the chimeric attenuated virus to the epidermal compartment of a subject's skin, preferably to the dendritic cells, using a device that disrupts the outer barrier layer of the skin, i.e., the stratum corneum. The invention encompasses methods for delivering the attenuated chimeric flavivirus vaccine to the skin of a subject, using devices that abrade the skin, particularly the stratum corneum. Devices and methods for delivery to the skin by abrasion are known in the art and encompassed within the invention, see, e.g., US Publication No. 2003/0093040, which is incorporated herein by reference in its entirety.

In some embodiments, devices used for epidermal delivery of the chimeric vaccines of the invention penetrate, but do not pierce, the stratum corneum. The chimeric vaccines to be administered using the methods of this invention may be applied to the skin prior to abrading, simultaneous with abrading, or post-abrading. In a preferred specific embodiment, chimeric flavivirus vaccines of the invention are applied to the skin simultaneously with abrasion rather than being applied to previously abraded skin. Although not intending to be bound by a particular mechanism of action, the efficacy of chimeric flaviviral vaccines are improved when they are abraded into the skin rather than being passively applied to skin which has been previously abraded. The methods of the invention thus result in a more protective immune response compared to conventional modes of delivery, i.e., syringe and needle method. Although not intending to be bound by a particular mechanism of action, the benefits and advantages of the methods of the invention are in part due to their ability to specifically target the epidermal compartment and the immune cells residing therein, e.g., dendritic cells and Langerhans cells.

Delivering the vaccine formulations of the invention to the epidermal compartment results in a longer duration of viremia and higher levels of antibody titre as determined using common methods known to one skilled in the art. Preferably the length of viremia is increased by about 2-fold, about 4-fold, about 8-fold. In a specific preferred embodiment, the length of the viremia is increased by about 2-fold. The level of antibody titre is preferably raised by about 2-fold, about 4-fold, about 8-fold.

The present invention provides devices and methods for abrading the stratum corneum to enhance delivery and specific epidermal targeting of the vaccine formulations of the invention. As used herein, the term “abrade” refers to removing at least a portion of the stratum corneum to increase the permeability of the skin without causing excessive skin irritation or compromising the skin's barrier to infectious agents. This is in contrast to “puncturing” which produces discrete holes through the stratum corneum with areas of undisrupted stratum corneum between the holes.

In some embodiments, the microabrader used in the methods of the invention is a device capable of abrading the skin to increase the permeability of the skin without causing excessive skin irritation or compromising the skin's barrier to infectious agents. In preferred embodiments, the device is capable of abrading the skin thereby penetrating the stratum corneum without piercing the stratum corneum. As used herein, “penetrating” refers to entering the stratum corneum without passing completely through the stratum corneum and entering into the adjacent layers. This is not to say that that the stratum corneum can not be completely penetrated to reveal the interface of the underlying layer of the skin. Piercing, on the other hand, refers to passing through the stratum corneum completely and entering into the adjacent layers below the stratum corneum.

In one preferred embodiment, the microabrader also includes an effective amount of the chimeric vaccine to be delivered. By an “effective amount” of the chimeric vaccine is intended to mean an amount that will elicit a desired therapeutically effective response in a subject, including, but not limited to, an immunostimulatory or immunomodulatory response.

The microabrader devices used in the methods of the invention is believed to have a unique immunological advantage in the delivery of vaccines with the potential of increasing the vaccine's clinical value. The penetration of the multiple microprotrusions into the stratum corneum is suggested as having an adjuvant-like stimulatory effect. The “penetration” response from the multiple microprotrusion is believed more than a simple acute inflammatory response. These “penetration” effects can cause damage to a variety of cells and cellular architecture, causing the appearance of polymorphonuclear neutrophils (PMN) and macrophages as well as the release of IL1, tumor necrosis factor (TNF) and other agents, which can lead to a number of other immunological responses. The soluble stimulatory factors influence the proliferation of lymphocytes and are central to the immune response to vaccines. In addition, these factors influence the migration and activation of resident antigen presenting cells including Langerhans cells and dendritic cells. The microabrader of the present invention is valuable in promoting significant immune response to a vaccine in the abraded area. The small grooves and furrows created by the microprotrusion array over the abraded area are believed to increase the availability of the vaccine antigen for interaction with antigen-presenting cells compared to a vaccine applied topically in the absence of abrasion or administered using standard needles.

The formulations of the invention may be delivered into the skin in any pharmaceutically acceptable form. In one embodiment the vaccine formulation of the invention is applied to the skin and an abrading device is then moved or rubbed reciprocally over the skin. It is preferred that the minimum amount of abrasion be used to produce the desired result. Determination of the appropriate amount of abrasion is within the ordinary skill in the art. In another embodiment the vaccine formulation may be applied in dry form to the abrading surface of the delivery device prior to application. In this embodiment, a reconstituting liquid is applied to the skin at the delivery site and the vaccine-coated abrading device is applied to the skin at the site of the reconstituting liquid. It is then moved or rubbed reciprocally over the skin so that the vaccine becomes dissolved in the reconstituting liquid on the surface of the skin and is delivered simultaneously with abrasion. Alternatively, a reconstituting liquid may be contained in the abrading device and released to dissolve the vaccine as the device is applied to the skin for abrasion.

The microabrader devices used in the methods of the invention, comprise microprotrusion of variable length and thickness selected based on the thickness of the stratum corneum in the location where the device is to be applied. Preferably, the microprotrusions penetrate the stratum corneum substantially without piercing or passing through the stratum corneum. The microprotrusions can have a length up to about 500 microns. Suitable microprotrusions have a length of about 50 to 500 microns. Preferably, the microprotrusions have a length of about 50 to about 300 microns, and more preferably in the range of about 150 to 250 microns, with 180 to 220 microns most preferred. The microprotrusions have a generally pyramidal shape and are perpendicular to the plane of the device. These shapes have particular advantages in insuring that abrasion occurs to the desired depth. Guidelines for the design of microprotrusion is set forth in U.S. Publication No. 2003/009304, and U.S. Pat. No. 6,595,947, both of which are incorporated herein by reference in their entirety.

The delivery site of the skin is prepared so that the vaccine formulation is effectively and specifically targeted to the epidermal compartment. The microabrader is gently pressed against the skin and then moved over or across the skin. The length of the stroke of the microabrader can vary depending on the desired size of the delivery site, defined by the delivery area desired. The dimensions of the delivery site are selected to accomplish the intended result and can vary depending on the formulation, and the form of the formulation, being delivered. The microabrader is then lifted from the skin to expose the abraded area and a suitable delivery device, patch or topical formulation may be applied to the abraded area. Alternatively, the formulation to be administered may be applied to the surface of the skin either before, or simultaneously with abrasion.

The extent of the abrasion of the stratum corneum is dependent on the pressure applied during movement and the number of repetitions with the microabrader device. In one embodiment, the microabrader is lifted from the skin after making the first pass and placed back onto the starting position in substantially the same place and position. The microabrader is then moved a second time in the same direction and for the same distance. In another embodiment, the microabrader is moved repetitively across the same site in alternating direction without being lifted from the skin after making the first pass. Generally, two or more passes are made with the microabrader. In further embodiments, the microabrader can be swiped back and forth, in the same direction only, in a grid-like pattern, a circular pattern, or in some other pattern for a time sufficient to abrade the stratum corneum a suitable depth to enhance the delivery of the vaccine formulation.

In some embodiments, the invention encompasses delivering the chimeric flavivirus vaccines of the invention to the intradermal compartment of a subject's skin preferably by directly and selectively targeting the intradermal space. Although not intending to be bound by a particular mechanism of action, delivering the chimeric flavivirus vaccine formulations of the invention to the intradermal space improves the availability of the chimeric vaccine to the antigen presenting cells residing therein and enhances the antigen specific immune response. The chimeric flavivirus vaccine formulations of the invention are administered using any of the intradermal devices and methods disclosed in U.S. patent application Ser. No. 09/417,671, filed on Oct. 14, 1999; Ser. No. 09/606,909, filed on Jun. 29, 2000; Ser. No. 09/893,746, filed on Jun. 29, 2001; Ser. No. 10/028,989, filed on Dec. 28, 2001; Ser. No. 10/028,988, filed on Dec. 28, 2001; or International Publication No.'s EP 10922 444, published Apr. 18, 2001; WO 01/02178, published Jan. 10, 2002; WO 02/02179, published Jan. 10, 2002; and U.S. application Ser. No. 10/185,717 filed Jul. 2, 2002, all of which are incorporated herein by reference in their entirety.

The present invention provides a method to improve the availability of a chimeric flavivirus vaccine formulation of the invention to the immune cells residing in the skin, e.g., antigen presenting cells, in order to effectuate an antigen-specific immune response to the vaccine formulation by accurately targeting the intradermal space. Preferably, the methods of the invention, allow for smaller doses of the chimeric flavivirus vaccine formulation to be administered via the intradermal route. The intradermal methods of administration comprise conventional injection needles, catheters, microcannula or microneedles of all known types, employed singularly or in multiple needle arrays and infusion systems or any other means to accurately target the intradermal space. The intrademal methods of administration encompass not only microdevice-based injection means, but other delivery methods such as needle-free ballistic injection of fluids or powders into the intradermal space, Mantoux-type intradermal injection, enhanced ionotophoresis through microdevices, and direct deposition of fluid, solids, or other dosing forms into the skin.

The invention encompasses the use of conventional injection needles, catheters or microneedles of all known types, employed singularly or in multiple needle arrays. The terms “needle” and “needles” as used herein are intended to encompass all such needle-like structures. The term “microneedles” as used herein are intended to encompass structures smaller than about 30 gauge, typically about 31-50 gauge when such structures are cylindrical in nature. Non-cylindrical structures encompass by the term microneedles would therefore be of comparable diameter and include pyramidal, rectangular, octagonal, wedged, and other geometrical shapes. The intradermal delivery of the vaccine formulations of the invention may use ballistic fluid injection devices, powder jet delivery devices, piezoelectric, electromotive, electromagnetic assisted delivery devices, gas-assisted delivery devices, which directly penetrate the skin to directly deliver the vaccine formulations of the invention to the targeted location within the dermal space.

The actual method by which the intradermal vaccine formulations of the invention are targeted to the intradermal space is not critical as long as it penetrates the skin of a subject to the desired targeted depth within the intradermal space without passing through it. The actual optimal penetration depth will vary depending on the thickness of the subject's skin. In most cases, skin is penetrated to a depth of about 0.5-2 mm. Regardless of the specific intradermal device and method of delivery, the intradermal vaccine formulation preferably targets the vaccine formulations of the invention to a depth of at least 0.3 mm, more preferably at least 0.5 mm up to a depth of no more than 2.5 mm, more preferably no more than 2.0 mm, and most preferably no more than 1.7 mm.

In some embodiments, the vaccine formulations are delivered at a targeted depth just under the stratum corneum and encompassing the epidermis and upper dermis, e.g., about 0.025 mm to about 2.5 mm. In order to target specific cells in the skin, the preferred target depth depends on the particular cell being targeted and the thickness of the skin of the particular subject. For example, to target the Langerhans cells in the dermal space of human skin, delivery would need to encompass, at least, in part, the epidermal tissue depth typically ranging from about 0.025 mm to about 0.2 mm in humans.

In some embodiments, when the vaccine formulations require systemic circulation, the preferred target depth would be between, at least about 0.4 mm and most preferably, at least about 0.5 mm, up to a depth of no more than about 2.5 mm, more preferably, no more than about 2.0 mm and most preferably, no more than about 1.7 mm. Targeting the vaccine formulations predominately at greater depths and/or into a lower portion of the reticular dermis is usually considered to be less desirable.

The intradermal administration methods useful for carrying out the invention include both bolus and infusion delivery of the vaccine formulations to a subject, preferably a mammal, most preferably a human. A bolus dose is a single dose delivered in a single volume unit over a relatively brief period of time, typically less than about 10 minutes. Infusion administration comprises administering a fluid at a selected rate that may be constant or variable, over a relatively more extended time period, typically greater than about 10 minutes.

The intradermal delivery of the formulations into the intradermal space may occur either passively, without application of the external pressure or other driving means to the vaccine formulations to be delivered, and/or actively, with the application of pressure or other driving means. Examples of preferred pressure generating means include pumps, syringes, elastomer membranes, gas pressure, piezoelectric, electromotive, electromagnetic pumping, or Belleville springs or washers or combinations thereof. If desired, the rate of delivery of the intradermal vaccine formulations of the invention may be variably controlled by the pressure-generating means.

The vaccine formulations delivered or administered in accordance with the invention include solutions thereof in pharmaceutically acceptable diluents or solvents, suspensions, gels, particulates such as micro- and nanoparticles either suspended or dispersed, as well as in-situ forming vehicles of same.

5.4 Determination of Efficacy of Vaccine Formulations

The invention encompasses methods for determining the efficacy of the chimeric vaccine formulations using any standard method known in the art or described herein. The assay for determining the efficacy of the chimeric vaccine formulations of the invention may be in vitro based assays or in vivo based assays, including animal based assays.

The invention encompasses biochemical and/or immunochemical based assays to determine the rate of growth of a chimeric virus of the invention in a cell culture system, an animal model system or in a subject. For example Vero cells may be infected with a chimeric virus of the invention, and the growth of the virus may be determined using a plaque assay. In a specific embodiment, the viral titre is determined by infecting a monolayer of cells that are susceptible to infection with the virus at a dilution of the virus that allows for the emergence of single plaques. The plaques can then be counted and the viral titre expressed as plaque forming units per milliliter of sample. Plaque assays for determining viral growth of chimeric flaviviruses are known in the art and employ a double agarose overlay and neutral red assay, see, e.g., Monath et al., 2000, J. Virol. 74: 1742-51; Guirakhoo et al., 2001, J. of Virology, 75(16): 7290-7304; Guirakhoo et al., 2000, J. of Virol. 74(12): 5477-5485; all of which are incorporated herein by reference in their entirety.

An exemplary plaque assay that may be used to determine the growth of the chimeric viruses of the invention may comprise the following: providing human dendritic cells, which may be isolated from normal human peripheral blood by leukophoresis and density gradient centrifugation; infecting the DC's with wild-type dengue virus, Chimerivax or a commercial YF 17D vaccine; incubating the cells at 37° C., 5% CO₂; supplementing the cells with LGM-3 medium; culturing the cells at 37° C., for preferably 4 days; collecting cell free supernatants at certain time intervals post-infection (e.g., 8 hr, 24 hr, 48 hr, 72 hr, 96 hr); mixing the supernatants with heat inactivated FBS; measuring the virus titres in the DC supernatant for the control and the infected cells in a plaque assay using Vero cells by measuring the plaque size.

In a specific embodiment of the invention, the growth rate of a virus of the invention in a subject is estimated by the titre of antibodies against the virus in the subject. Without being bound by theory, the antibody titre in the subject reflects not only the viral titre in the subject but also the antigenicity. If the antigenicity of the virus is constant, the increase of the antibody titre in the subject can be used to determine the growth curve of the virus in the subject. In a preferred embodiment, the growth rate of the virus in animals or humans is best tested by sampling biological fluids of a host at multiple time points post-infection and measuring viral titre. In a specific embodiment, the viral titre is determined by obtaining a sample from the infected subject, preparing a serial dilution of the sample and infecting a monolayer of cells that are susceptible to infection with the virus at a dilution of the virus that allows for the emergence of single plaques. The plaques can then be counted and the viral titre express as plaque forming units per milliliter of sample.

The invention encompasses determining the prophylactic and therapeutic efficacy of a vaccine formulation of the invention in a subject who has been administered a formulation of the invention, by measuring viremia and neutralizing antibody response. Methods for determining viremia and neutralizing antibody response to a flavivirus are known in the art and encompasses within the instant invention. Preferably, prior to immunization with a vaccine formulation of the invention, a control sample, blood, is obtained from the subject; then following immunization viremia and neutralizing antibody response are monitored serially, preferably daily. In some embodiments, viremia and neutralizing antibody response is determined on about days 2-11 post immunization day, i.e., day 1, and neutralizing antibody response is determined on about day 31 and about day 61 post-immunization. Viremia and plaque reduction neutralization test are preferably determined on Vero cells, using agarose double overlay and neutral red assays as described previously, see, e.g., Monath et al., 2000, J. Virol. 74: 1742-51; Guirakhoo et al., 2001, J. of Virology, 75(16): 7290-7304; Guirakhoo et al., 2000, J. of Virol. 74(12): 5477-5485; all of which are incorporated herein by reference in their entirety. In some embodiments, viral titres are determined by direct plaquing on Vero cells using undiluted, and diluted samples, e.g., 2-fold and 10-fold dilutions of the sample. Neutralizing antibody titres are preferably determined on heat inactivated sera without the addition of complement. Methods for determined neutralizing antibody response are known in the art, see, e.g., Monath et al., 2000, J. Virol. 74: 1742-51.

In some embodiments, the invention encompasses detecting and/or quantitating a humoral immune response against the antigenic or immunogenic agent, e.g., the prM-E protein of a flavivirus, of a vaccine formulation of the invention in a sample, e.g., serum, obtained from a subject who has been administered a vaccine formulation of the invention. Preferably, the humoral immune response of the vaccine formulations of the invention are compared to a control sample obtained from the same subject, who has been administered a control formulation, e.g., a formulation which simply comprises of the antigenic or immunogenic agent.

Assays for measuring humoral immune response are well known in the art, e.g., see, Coligan et al., (eds.), 1997, Current Protocols in Immunology, John Wiley and Sons, Inc., Section 2.1. A humoral immune response may be detected and/or quantitated using standard methods known in the art including, but not limited to, an ELISA assay. Preferably, the humoral immune response is measured by detecting and/or quantitating the relative amount of an antibody which specifically recognizes an antigenic or immunogenic agent in the sera of a subject who has been treated with a vaccine formulation of the invention relative to the amount of the antibody in an untreated subject. ELISA assays can be used to determine total antibody titres in a sample obtained from a subject treated with a formulation of the invention. In other embodiments, ELISA assays may be used to determine the level of isotype specific antibodies using methods known in the art.

ELISA based assays comprise preparing an antigen, coating the well of a 96 well microtitre plate with the antigen, adding an antibody specific to the antigen conjugated to a detectable compound such as an enzymatic substrate (e.g., horseradish peroxidase or alkaline phosphatase) to the well and incubating for a period of time, and detecting the presence of the antigen. In an ELISA assay, the antibody does not have to be conjugated to a detectable compound; instead, a second antibody (which recognizes the first antibody) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 11.2.1.

In other embodiments, a T cell immune response may be measured, for example by measuring cytokine production using common methods known to one skilled in the art including but not limited to ELISA from tissue culture supernatants, flow cytometry based intracellular cytokine staining of cells ex vivo or after an in vitro culture period, and cytokine bead array flow cytometry based assay. In yet other embodiments, the invention encompasses measuring T cell specific responses using common methods known in the art, including but not limited to chromium based release assay, flow cytometry based tetramer or dimer staining assay using known CTL epitopes.

5.5 Prophylactic and Therapeutic Uses

The invention provides methods of treatment and prophylaxis which involve administering a chimeric flavivirus vaccine formulation of the invention to a subject, preferably a mammal, and most preferably a human for treating, managing or ameliorating symptoms associated with a flavivirus infection, e.g., Dengue virus, Japanese Encephalitis. The subject is preferably a mammal such as a non-primate, e.g., cow, pig, horse, cat, dog, rat, and a primate, e.g., a monkey such as a Cynomolgous monkey and a human. In a preferred embodiment, the subject is a human.

The invention encompasses a method for immunization and/or stimulating an immunological immune response in a subject comprising delivery of a single dose of an chimeric vaccine formulation of the invention to a subject, preferably a human. In some embodiments, the methods of the invention specifically target the epidermal compartment. In a specific embodiment, the methods of the invention use needleless devices. In yet other embodiments, the methods of the invention encompass delivering the chimeric vaccine formulations of the invention to the intradermal compartment of a subject's skin. In some embodiments, the invention encompasses one or more booster immunizations. The vaccine formulation of the invention is particularly effective in stimulating and/or upregulating an antibody response to a level greater than that seen in conventional vaccine formulations and administration schedules. For example, a vaccine formulation of the invention may lead to an antibody response comprising generations of one or more antibody classes, such as IgM, IgG, and/or IgA. Most preferably, the vaccine formulations of the invention stimulate a systemic immune response that protects the subject from at least one pathogen. The vaccine formulations of the invention may provide systemic, local, or mucosal immunity or a combination thereof.

5.6 Kits

The invention also provides a pharmaceutical pack or kit comprising an chimeric flavivirus vaccine formulation of the invention and a device for delivery of the chimeric vaccine in accordance with the methods of the invention. In a specific embodiment the invention provides a kit comprising, one or more containers filled with one or more of the components of the chimeric vaccine formulation of the invention. Associated with such container(s) can be a notice in the form prescribed by a governmental agency regulating the manufacture, use or sale of pharmaceuticals or biological products, which notice reflects approval by the agency of manufacture, use or sale for human administration. In a specific embodiment, the invention comprises kits comprising a device for epidermal delivery and a chimeric flavivirus vaccine formulation of the invention as described herein. In another specific embodiment, the invention comprises kits comprising a device for intradermal delivery and a chimeric flavivirus vaccine formulation of the invention as described herein.

In a specific embodiment, the invention provides a kit for use in inducing an immune response to a flavivirus viral antigen in a subject, said kit comprising: (a) a chimeric yellow fever virus expressing an envelope gene product of a flavivirus, e.g., Dengue, JE, and (b) a device, e.g., a microabrader device, that abrades an epidermal layer of the skin's surface while delivering the chimeric virus. In another specific embodiment, the invention provides a kit for use in inducing an immune response to a flavivirus viral antigen in a subject, said kit comprising: (a) a chimeric yellow fever virus expressing an envelope gene product of a flavivirus e.g., Dengue, JE and (b) a device that that targets the intradermal compartment of the subject's skin.

### 6. EXAMPLES

6.1 In Vitro Infection of Human Dendritic Cells with Chimerivax™-Dengue and Wild-Type Dengue Viruses

The objective of this study was to determine whether dendritic cells are permissive to ChimeriVax™ virus infection.

Methods

DC culture. Normal Human Dendritic Cells (NHDC) were purchased from Cambrex Bio Science Walkersville, Inc. DCs had been isolated from normal human peripheral blood from the same donor by leukopheresis and density gradient centrifugation, and then cryopreserved. Twenty-four hours prior to infection, cells were thawed, counted, and plated into T25 flasks at a density of 3.4×10⁴ cells/cm². Cells were grown in LGM-3 medium (Lymphocyte Growth Medium 3, Clonetics, Cambrex Bio Science Walkersvile, Inc) and supplemented with rIL-4 and rGM-CSF (R&D Systems, Inc. Birmingham) to yield a final concentration of 500 units/ml.

Infection of DCs. DCs were infected with wild type dengue parent viruses; DEN-1 PUO 359 (TVP-1140), DEN-2 PUO 218, DEN-3 PaH881/88, DEN-4 1228 (TVP 980), ChimeriVax™ viruses; ChimeriVax-DEN1, ChimeriVax-DEN2, ChimeriVax-DEN3, ChimeriVax-DEN4, or commercial YF 17D vaccine (YF-VAX®, Aventis Pasteur Lyon, France). Cells were infected at a multiplicity of infection (MOI) of 0.168 for DEN-1 PUO 359 (TVP-1140) and ChimeriVax-DEN1 viruses, and at an MOI of 0.074 for all other viruses. After 1-h incubation at 37° C., 5% CO₂, 6.5 ml of rIL-4 and rGM-CSF supplemented LGM-3 medium was added to each flask. DCs were cultured at 37° C., 5% CO₂, for 4 days. At 8 h, 24 h, 48 h, 72 h, and 96 h, cell-free supernatants were collected, mixed with equal volume of heat inactivated FBS, and stored at −70° C.

Virus control at 37° C. 5% CO₂. Since the DCs were not washed after infection to remove unbound viruses, it was necessary to include a virus control in the experiment to assure that infection and replication of viruses in DCs is real and not due to the presence of inocula in the supernatants. Control viruses (without DCs), at the same concentration as used for the DCs' infection, were incubated in 24 well plates at 37° C., 5% CO₂, for 4 days. At 8 h, 24 h, 48 h, 72 h, and 96 h, samples were collected, mixed with equal volume of heat inactivated FBS, and stored at −70° C.

Sample collection. 0.5 ml of DC supernatants was collected at 8 h, 24 h, 48 h, 72 h, and 96 h post infection. Since the DC cultures are loosely adherent, 0.5 ml of DC supernatants was collected in centrifuge tubes, and pelleted at 1,200 rpm for 5 minutes. The supernatant was mixed with equal volume of heat inactivated FBS, and stored at −70° C. The pellet was suspended in 0.5 ml of rIL-4 and rGM-CSF supplemented LGM-3 medium, and placed back into the corresponding flasks. For sample collection of the virus control, 100 μL of each virus was collected at 8 h, 24 h, 48 h, 72 h, and 96 h post-infection, mixed with equal volume of heat inactivated FBs, and stored at −70° C. After each sample collection, 100 μL of rIL-4 and rGM-CSF supplemented LGM-3 medium was dispensed into each well.

Plague assays. The DC supernatant virus titres and the virus control titres at 8 h, 24 h, 48 h, 72 h, and 96 h post infection were determined by plaque assay using Vero cells. Chimeric viruses and the YF 17D vaccine replicate efficiently in Vero cells and produce relatively large plaques (1-2 mm in diameter). Thus, a standard plaque assay using a double agarose overlay and neutral red was used for these viruses. In contrast, wild type dengue viruses produce extremely small plaques (<1 mm in diameter) in Vero cells, making it difficult to count plaques after the addition of neutral red. For this reason, wild type dengue virus titres were measured in an immunofocus assay using DEN specific antibodies (1:1,000 dilution of ascites D-2-1F1-3 antibodies, 1:4,000 dilution of den-2 3H5 antibodies for DEN-2 WT, 1:500 dilution of D6-8H1-12 antibodies for DEN-3 WT, and 1:1,000 dilution of ascites IH10 antibodies for DEN-4 WT), followed by alkaline phosphatase-conjugated goat anti-mouse IgG (See, Guirakhoo et al., 2001, J. Virol., 75: 7290-7304).

Results

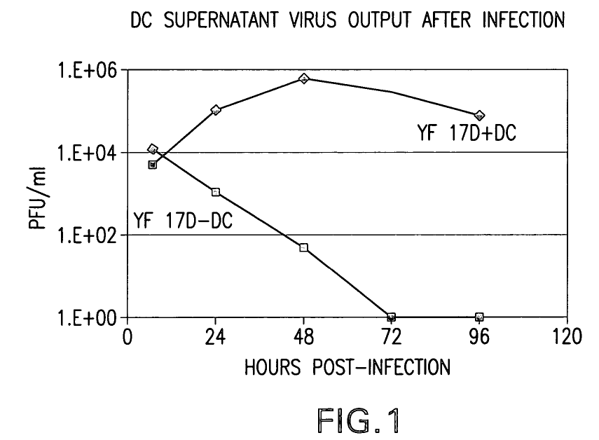

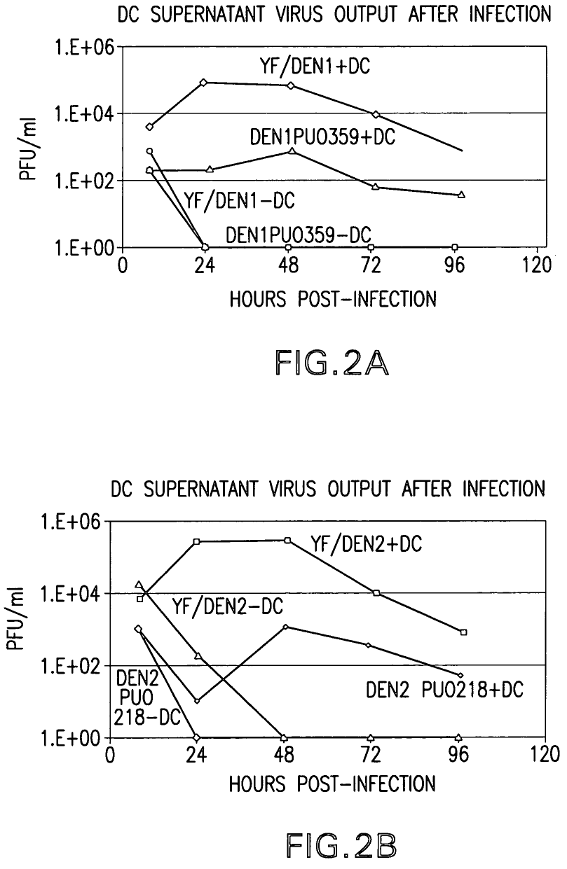

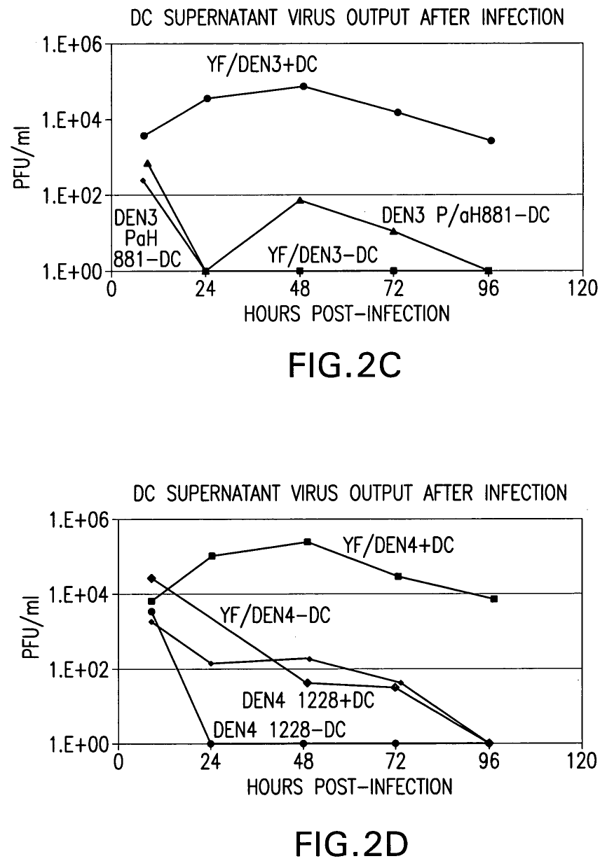

Virus replication in human blood-derived DCs. Since ChimeriVax™-dengue viruses had been constructed by incorporating prME genes of dengue wild-type viruses into core and non-structural genes of YF 17D virus, it was necessary to compare replication of ChimeriVax™ dengue viruses along with that of their parent viruses (YF 17D and dengue). The titre of YF 17D virus increased by nearly 2 log₁₀ PFU/ml after 48 hrs incubation with DCs. In contrast, the titre of virus in the absence of DCs dropped by more than 2 logs (from 4 to 2 log₁₀ PFU/ml) within the same time period (FIG. 1). The virus outputs from DCs infected with ChimeriVax™-DEN1-4 viruses were more or less similar to those of YF 17D virus, which were significantly higher than the virus outputs of their parent dengue viruses (FIG. 2).

6.2 Administration of Chimerivax™-JE™ Vacclne Using Subcutaneous Injection via Syringe, Intradermal Injection via Microneedle Single Cannula Device, and Epidermal Delivery via Microabrader Device:

6.2.1 Summary and Objectives

A Comparative Immunozenicity Study in Cynomolzus Monkey: A study was conducted to evaluate the feasibility of using Becton Dickinson microneedle (also referred to as MicroMedica) and microabrader (also referred to as OnVax) device in the delivery of Acambis ChimeriVax™-Japanese encephalitis (JE) vaccine in an appropriate animal model [non-human primates (cynomolgus monkeys)]. Safety and immunogenicity were determined by induction of viremia and immune responses at levels sufficient to protect the animals from an infectious challenge.

Twelve monkeys were divided into 4 groups (n=3), and each group was inoculated with 1.26×10⁴ PFU of ChimeriVax™-JE cGMP vaccine using various routes/devices: Group 1. Subcutaneous/syringe; Group 2. Intradermal/MicroMedica Single Cannula; Group 3. Epidermal/OnVax device, with pre-abrasion, and Group 4. Epidermal/OnVax device, with abrasion by the inoculum syringe.

Viremia was determined on Days 2 to 11, and neutralizing antibody response was measured on Day 31 post immunization. All monkeys in Groups 2 and 4 became viremic, whereas only ⅓ of animals in Groups 1 and 3 showed viremia. The duration, but not the magnitude, of viremia in Group 4 (Epidermal/OnVax) was significantly higher (p=0.01) than in Group 1 (Subcutaneous/syringe). The results of the measurement of neutralizing response, will show whether the extended duration of viremia in Group 4 results in a higher level of neutralizing antibody response and is presented herein.

In a separate experiment, blood-derived human dendritic cells were infected with ChimeriVax™ viruses to model the role of skin dendritic cells in the initiation of infection. Our findings suggest that dendritic cells are more permissive to Yellow Fever 17D and ChimeriVax™ dengue viruses than to parent wild-type dengue viruses. The OnVax device would be an excellent choice for efficient delivery of ChimeriVax™-JE, other ChimeriVax™ vaccine viruses, other flavivirus vaccines, or any vaccine viruses that specifically target dendritic cells of skin.

Objectives The objectives of this study were: (1) To evaluate the feasibility of using cynomolgus monkeys to determine the investigative delivery route/device combinations described below, and (2) To compare the viral replication (duration and magnitude of viremia) and immune response (neutralizing antibody production) in cynomolgus monkeys after administration of ChimeriVax-JE™ vaccine via the following delivery route/device combination:

1. Subcutaneous/syringe

2. Intradermal/MicroMedica Single Cannula

3. Epidermal/OnVax device, with pre-abrasion, and

4. Epidermal/OnVax device, with abrasion through the inoculum

6.2.2 Materials and Methods

Monkeys. A total of 12 (6 males and 6 females) experimentally-naïve, flavivirus-seronegative cynomolgus monkeys were assigned to treatment groups as shown in Table 1.

TABLE 1
EXPERIMENTAL GROUPS

| | | | | Dose | Dose |
|---|---|---|---|---|---|
| Group | Number | Delivery | Vaccine | Level | Volume |
| No. | of M/F | Route/Device | Preparation | (˜pfu^{a}) | (mL) |
| 1 | 2/1 | subcutaneous/ | ChimeriVax- | 1.26 × 10⁴ | 0.1 |
| | | syringe | JE ™ | | |
| 2 | 1/2 | intradermal/ | Vaccine | | |
| | | MicroMedica ® | | | |
| | | Single Cannula | | | |
| 3 | 2/1 | epidermal/ | | | |
| | | OnVax ® | | | |
| | | device, with pre- | | | |
| | | abrasion | | | |
| 4 | 1/2 | epidermal/ | | | |
| | | OnVax ® | | | |
| | | device, with | | | |
| | | abrasion | | | |
| | | through the | | | |
| | | inoculum. | | | |

^{a}pfu = plaque-forming units

All monkeys were dosed once on Day 1 as described above. The monkeys were evaluated for clinical signs of toxicity (twice daily) and for changes in body weight (weekly).

Blood samples were collected for: Determination of viremia on Days 2-11, and Determination of serum neutralizing antibody concentration; predose on Day 1, and on Day 31.

Vaccine. ChimeriVax-JE™ Vaccine (Part No. FP-0001, Lot Number 00C02); prepared in Vero (LS10) cells by BioReliance Corp. (Rockville, Md.), using GMP-complaint procedures; stabilized in modified Eagle medium with Earle's modified salts (MEME), 7.5% lactose and 2.5% humans serum albumin buffer. Titres were determined in Vero cells, and expressed as pfu/mL as 1.26×10⁶ pfu/mL.

Immunization. Monkeys were dosed by Group. Each dose (0.1 mL) of vaccine was administered on Day 1. Dosing sites were shaved and similarly prepared for all dose applications. Dosing sites were clearly marked. The animals were anesthetized with ketamine/dormitor, according to SBi SOP for dosing regardless of the dosing method. Anesthesia lasted for at least 30 minutes after the dosing was complete to allow sufficient time for the topically applied vaccine to air dry on the skin surface.

Route 1: Subcutaneous injection to Group 1 Each dose (0.1 mL) of vaccine was administered via subcutaneous injection into two sites on the same arm (0.05 mL per site). Disposable sterile syringes were used for each animal/dose.

Route 2: Intradermal administration to Group 2 Each dose (0.1 mL) of vaccine was administered via MicroMedica Single Cannula in two sites on the same arm. (0.05 mL per site). Disposable sterile syringes were used for each animal/dose.

Route 3: Epidermal administration to Group 3 Each dose (0.1 mL) of vaccine was administered topically to shaved skin that had been pre-abraded with OnVax device (consisting of a PMMA (polymethylmethacrylate) 1 cm² area abrading surface mounted to a hand-held polycarbonate applicator device as described in U.S. Pat. No. 6,595,947 and U.S. Publication No. 2003/0093040). The treatment process consisted of 6 successive passes of the device over the same location (3 passes in each direction). The 2 administration sites were located on the same shoulder/upper arm of each animal. Each administration site measured ˜1×2 cm, and received a total volume of 0.05 mL. The dosage volume was applied topically to the abraded skin, using a standard micro-pipettor fitted with a disposable sterile tip. The dosage was spread evenly over the treatment area using the side of the sterile pipette tip. Administration sites were left undisturbed and uncovered for at least 30 minutes to allow sufficient time for the vaccine to air dry completely.

Route 4: Epidermal administration to Group 4 Each dose (0.1 mL) or dosing suspension was administered topically to shaved skin, using OnVax device to abrade through the vaccine inoculum. The 2 administration sites were located on the same shoulder/upper arm of each animal. Each administration site measured ˜1×2 cm, and received a total volume of 0.05 mL. The dosage volume was applied topically to the shaved skin, using a standard micro-pipettor fitted with a disposable sterile tip. After topical application, the OnVax device as described above was placed in contact with the vaccine, and abraded through the inoculum. As above the treatment process consisted of 6 successive passes of the device over the same location (3 passes in each direction). Administration sites were left undisturbed and uncovered for at least 30 minutes to allow sufficient time for the vaccine to air dry completely.

In-Life Observations and Measurements. Procedures described in this section were applied to all monkeys on study, unless indicated otherwise.

(1) Cageside Observations

Frequency: Twice daily (a.m. and p.m.), from Day −5 to Day 31.

Procedure: Each monkey was observed for changes in general appearance or behavior, using a detailed checklist (per SBi SOP).

(2) Injection Site Observations

Frequency: Immediately after injection and at 0.5, 24, and 48 hours post injection.

Procedure: A digital photograph has been taken of each injection site at the designated time points above. Additionally, in Groups 1 (subcutaneous injection) and 2 (intradermal injection), the diameter of the “wheal” or fluid bleb just under the skin surface was measured with a ruler to the nearest millimeter.

(3) Feeding

Frequency: Once daily from Day −5 to Day 31, as part of the routine morning cageside Observations.

Procedure: The number of biscuits remaining from the previous day's feeding was examined, and a notation was made if less than approximately half of the rations was consumed.

(4) Body Weight Measurements

Frequency: Prior to the first dose (Day −1) and once weekly thereafter.

Procedure: Per SBi SOP. Food was withheld before body weights were measured.

(5) Viremia.

Viremia was determined in Vero cells using undiluted, 1:2 and 1:10 dilutions of sera, respectively, obtained on Days 2-11 post immunization as described previously (9).

6.2.3 Results and Discussion

Monkey experiment. Twelve cynomolgus monkeys were immunized with ChimeriVax™-JE cGMP vaccine using various routes/devices. The subcutaneous route was chosen as the reference route, because ChimeriVax™-JE vaccine is currently administered subcutaneously in humans. The additional routes of inoculation and devices were chosen for comparison purposes, because these routes and devices have shown initial enhancement of immunity in rats, which did not respond immunologically to inoculation of ChimeriVax™-JE when given subcutaneously. The dose levels were chosen after consideration of clinical dose levels of ChimeriVax™-JE, and evaluation of results from earlier studies in rhesus monkeys.

Viremia was determined on Days 2-11 post immunization. All 6 monkeys in Groups 2 and 4 became viremic, whereas only ⅓ of animals in Groups 1 and 3 showed viremia (Table 2).

TABLE 2
VIREMIA RESULTS: Viremia in cynomolgus
monkeys immunized with ChimeriVax ™-JE
cGMP vaccine* delivered by different routes/devices.

| | Delivery | | | | | | | | | | |
|---|---|---|---|---|---|---|---|---|---|---|---|
| | Route/ | Viremia by day post immunization Day | | | | | | | | | |
| Monkey | device | 2** | 3 | 4 | 5 | 6 | 7 | 8 | 9 | 10 | 11 |
| F21555M | ID/Micro- | 1.0 | 1.8 | 1.7 | — | — | — | — | — | — | — |
| F22685F | media, | — | — | — | — | — | — | — | — | — | — |
| F22452M | Single | — | — | — | — | — | — | — | — | — | — |
| F22163F | ID/Micro- | 1.5 | 1.7 | — | — | — | — | — | — | — | — |
| F22110M | media, | — | — | — | 1.7 | 1.0 | — | — | 4.3 | 1.0 | 0 |
| F21103F | Single | 2.2 | 2.3 | 2 | 1.5 | — | 1.0 | — | — | — | — |
| | Canula | | | | | | | | | | |
| F21729M | ED/ | — | — | — | — | — | — | — | — | — | — |
| F19966F | OnVax | — | — | — | — | — | — | — | — | — | — |
| F21517M | with pre- | — | — | 1.0 | 1.0 | 1.0 | 1.8 | 1.7 | 1.7 | 1.0 | — |
| | abrasion | | | | | | | | | | |
| F22162F | ED/ | — | — | — | 1.8 | 1.5 | 1.3 | 1.7 | 2.0 | 1.8 | 1.3 |
| F22112M | OnVax | — | — | — | 1.3 | 1.0 | 2.3 | 2.0 | 1.6 | 1.0 | — |
| F22166F | with | — | — | 1.7 | 1.6 | 1.8 | 1.6 | 1.8 | — | — | — |
| | abrasion | | | | | | | | | | |
| | through | | | | | | | | | | |
| | inoculum | | | | | | | | | | |

*4.1 log10 PFU was delivered in .
**Vaccine was administered on Day 1;
***No virus was detected with undiluted 1:2 or 1:10 dilution of sera; level of detection = 1 Jog10 PFU/ml

The mean peak viremias were 1.8, 1.9, 1.8 and 2.0 log₁₀ PFU/ml for Groups 1-4, respectively. The mean duration of viremia was 3, 3.7, 7, and 6 days for Groups 1-4, respectively (Table 3).

TABLE 3
SUMMARY OF VIREMIA

| | | Viremia | |
|---|---|---|---|
| Group | Delivery Route/Device | Mean Peak | Mean Duration |
| 1 | subcutaneous/syringe | 1.8 | 3 |
| 2 | intradermal/MicroMedica ® | 1.9 | 3.7 |
| | Single Cannula | | |
| 3 | epidermal/OnVax ® device, | 1.8 | 7 |
| | with pre-abrasion | | |
| 4 | epidermal/OnVax ® device, | 2.0 | 6 |
| | with abrasion through the | | |
| | inoculum. | | |

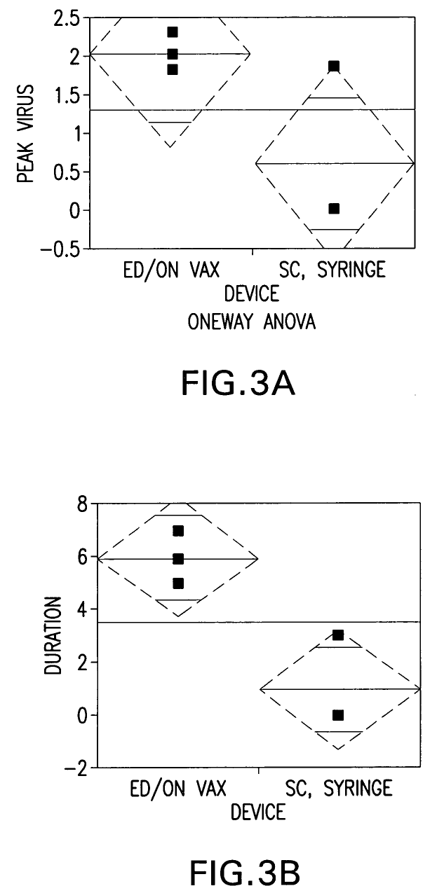

It appears that the duration, but not the magnitude, of viremia in Groups 3 and 4 was significantly higher than in Groups 1 and 2 (p=0.08 for magnitude of viremia, and p=0.01 for duration of viremia, in comparison t-tests for Groups 1 and 4) (FIG. 3). The measurement of neutralizing responses is shown below in Table 4.

TABLE 4
NEUTRALIZING ANTIBODY RESPONSE

| | | Nab Titer | | |
|---|---|---|---|---|
| Monkey | Delivery | d1 | d31 | d61 |
| F21555M | SC | <10 | 640 | 80 |
| F22685F | | <10 | 1,280 | 640 |
| F22452M | | <10 | 1,280 | 640 |
| F22163F | ID (mMedica) | <10 | 10,240 | 1,280 |
| F22110M | | <10 | 1,280 | 1,280 |
| F21103F | | <10 | 10,240 | 2,560 |
| F21729M | ED (OnVax | <10 | 160 | 20 |
| F19966F | pre-abrasion) | <10 | <10 | <10 |
| F21517M | | <10 | 10,240 | 640 |
| F22162F | ED (OnVax | <10 | 160 | 80 |
| F22112M | abrade thru) | <10 | 10,240 | 320 |
| F22166F | | <10 | 2,560 | 1,280 |

SUMMARY: In summary, it appears that the delivery of ChimeriVax™-JE by the epidermal route using OnVax device results in longer duration of viremia in monkeys. The magnitude of viremia, which generally correlates with the severity of dengue disease, is not affected by the route/delivery device. The duration of viremia in Groups 3 and 4 was similar. However, only ⅓ of animals in Group 3 became viremic, indicating that abrasion through inocula may have been more efficient in the delivery of the vaccine virus to the skin cells than pre-abrasion. The growth kinetic studies of ChimeriVax™ dengue viruses in human DCs indicate a possibility of skin DCs being the initial target of these viruses delivered by OnVax device. We are currently evaluating the neutralizing antibody responses against JE virus of the monkeys in this study. This response level will be a major contributor in the determination of the protection against the disease as well as the correlation between longer duration of viremia and higher magnitude of JE specific neutralizing antibody responses. If this proves to be correct, OnVax device will be an effective tool for the delivery of vaccines that target dendritic skin cells as entry route.

The invention described and claimed herein is not to be limited in scope by the specific embodiments herein disclosed since these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout this application various publications are cited. Their contents are hereby incorporated by reference into the present application in their entireties for all purposes.

What is claimed is:

1. A method for inducing an immune response to a flavivirus viral antigen in a subject, comprising delivering a chimeric yellow fever virus expressing an envelope gene product of a flavivirus virus to a subject's skin using a device that abrades an epidermal layer of the skin's surface while delivering the chimeric virus.

2. The method of claim 1, wherein the flavivirus is Japanese encephalitis.

3. The method of claim 1, wherein the flavivirus is Dengue.

4. The method of claim 1, wherein the chimeric virus is delivered to an epidermal compartment of the subject's skin.

5. The method of claim 1, wherein the chimeric virus is delivered to an immune cell in the subject's skin.

6. The method of claim 5, wherein the immune cell is a dendritic cell or a Langerhan cell.

7. The method of claim 1, wherein the device is needleless.

8. The method of claim 1, wherein the device is a microabrader device.

9. The method of claim 1, wherein the device comprises at least one microneedle.

10. The method of claim 1 wherein the subject is a human.

11. A kit for use in inducing an immune response to a flavivirus viral antigen in a subject, said kit comprising: (a) a chimeric yellow fever virus expressing an envelope gene product of a flavivirus and (b) a device that abrades an epidermal layer of the skin's surface while delivering the chimeric virus.

12. The kit of claim 11, wherein the device is needleless.

13. The kit of claim 11, wherein the device is a microabrader device.

14. The kit of claim 11, wherein the device comprises at least one microneedle.

15. The kit of claim 11, wherein the flavivirus is Japanese Encephalitis or Dengue.
